(19) United States
(12) Patent Application Publication
(10) Pub. No.: US 2026/0069545 A1
(43) Pub. Date: Mar. 12, 2026

(54) MODIFIED RELEASE FORMULATIONS OF IBUDILAST

(71) Applicant: MEDICINOVA, INC., La Jolla, CA (US)

(72) Inventor: Kazuko Matsuda, La Jolla, CA (US)

(73) Assignee: MEDICINOVA, INC., La Jolla, CA (US)

(21) Appl. No.: 19/325,985

(22) Filed: Sep. 11, 2025

Related U.S. Application Data
(60) Provisional application No. 63/693,377, filed on Sep. 11, 2024.

(51) Int. Cl. A61K 9/48; A61K 9/16; A61K 9/50; A61K 31/437

(52) U.S. Cl. CPC A61K 9/4866; A61K 9/1611; A61K 9/1623; A61K 9/1652; A61K 9/4858; A61K 9/501; A61K 9/5015; A61K 9/5026; A61K 9/5042; A61K 9/5084; A61K 31/437

(57) ABSTRACT

Described herein are pharmaceutical compositions comprising extended-release pellets and optionally delayed release pellets, wherein each of the extended-release pellets and each of the delayed release pellets comprises a drug pellet and an outer coating; the outer coating of the extended-release pellets is an extended-release outer coating; the outer coating of the delayed release pellets is a delayed release outer coating; the outer coating of the extended-release pellets is an extended-release outer coating; the outer coating of the delayed release pellets is a delayed release outer coating; the drug pellet comprises about 15 wt. % to about 25 wt. % ibudilast, about 60 wt. % to about 70 wt. % sugar sphere, about 5 wt. % to about 10 wt. % hydroxypropyl methylcellulose, and about 4 wt. % to about 8 wt. % silicon dioxide; the extended-release outer coating comprises about 20 wt. % to about 55 wt. % ethyl cellulose, about 20 wt. % to about 55 wt. % hydroxypropyl methylcellulose, about 1 wt. % to about 4 wt. % triethyl citrate, and about 20 wt. % to about 25 wt. % talc, wherein the weight percentages of ethyl cellulose, hydroxypropyl methylcellulose, triethyl citrate, and talc are based on total weight of the extended-release outer coating; and the delayed release outer coating comprises about 55 wt. % to about 65 wt. % copolymer of methacrylic acid and ethyl acrylate, about 3 wt. % to about 9 wt. % triethyl citrate, and about 25 wt. % to about 35 wt. % talc, wherein the weight percentages of the copolymer of methacrylic acid and ethyl acrylate, triethyl citrate, and talc are based on total weight of the delayed release outer coating.

### CROSS-REFERENCE TO RELATED APPLICATIONS

This application claims the benefit of priority to U.S. Provisional Application No. 63/693,377, filed on Sep. 11, 2024, the contents of which are incorporated by reference herein in their entirety.

### BACKGROUND

The small molecule ibudilast (3-isobutyryl-2-isopropylpyrazolo[1,5-a]pyridine) is an inhibitor of macrophage inhibitory factor (MIF) (Cho et al., PNAS-USA, 2010 June 107:11313-8), is a selective inhibitor of cyclic nucleotide phosphodiesterases (PDEs) 3A, 4, 10A1 and 11A1 (Gibson et al., Eur. J. Pharmacol., 538:39-42, 2006), and has toll-like receptor-4 (TLR4) antagonistic activity (Yang et al., Cell Death and Disease (2016) 7, e2234; doi: 10.1038/cddis.2016.140). Ibudilast distributes well to the CNS (Sanftner et al., Xenobiotica, 2009 39:964-977) and at clinically-relevant plasma or CNS concentrations, ibudilast selectively inhibits macrophage migration inhibitory factor (MIF) and, secondarily, PDEs 3, 4, 10 and 11. Ibudilast also acts as a leukotriene D4 antagonist, an anti-inflammatory, a PAF antagonist, and a vasodilatory agent (Thompson Current Drug Reports). Ibudilast is thought to exert a neuroprotective role in the central nervous system of mammals, presumably via suppression of the activation of glial cells (Mizuno et al., Neuropharmacology 46:404-411, 2004).

Ibudilast has been widely used in Japan for relieving symptoms associated with ischemic stroke or bronchial asthma. In recent clinical trials, its use in the treatment of multiple sclerosis (MS), an inflammatory disease of the central nervous system, has been explored (News. Medical.Net; Pharmaceutical News, 2 Aug. 2005). As disclosed in this publication, this clinical trial was expected to treat “relapsing-remitting MS,” however, no mention is made of progressive multiple sclerosis. In U.S. Pat. No. 6,395,747, ibudilast is disclosed as a treatment for multiple sclerosis, which is generally understood to mean relapsing and remitting multiple sclerosis, not progressive multiple sclerosis. U.S. Patent Application Publication No. 20060160843 discloses ibudilast for the treatment of intermittent and short term pain, however, this is not pain related to a progressive neurodegenerative disease. However, U.S. Pat. No. 9,314,452 discloses ibudilast as a treatment for amyotrophic lateral sclerosis, a progressive neurodegenerative disease. Similarly, U.S. Pat. No. 8,138,201 discloses ibudilast as a treatment for primary progressive multiple sclerosis and/or secondary progressive multiple sclerosis.

An oral “intermediate” release formulation of ibudilast (10 mg/capsule) was previously commercially available. However, multiple capsules were still required to achieve expected efficacy exposure, often requiring administration 2-3 times per day with a 60-100 mg (i.e., 6-10 capsules) daily dosage, and frequently reported side effects of its administration included gastrointestinal symptoms such as nausea, loss of appetite, loose stool and diarrhea. An oral ibudilast formulation to address patient compliance and better tolerance is needed.

### SUMMARY

Provided herein in one aspect is a pharmaceutical composition comprising ibudilast as active agent, wherein desired active agent loading is adjusted in each capsule by varying amounts of extended-release pellets and optionally delayed-release pellets present in the composition. In some embodiments, each capsule contains 20-50 mg, and the pharmaceutical composition may therefore be daily administered as fewer capsules per day (e.g., 1 or 2 capsules, with each capsule containing the extended-release pellets and optionally the delayed release pellets). The pharmaceutical compositions described herein exhibit a higher Tₘₐₓ value when administered with food (e.g., immediately before or after meal), which is a realistic scenario in clinical use, compared to the Tₘₐₓ value of the intermediate release composition, while maintaining similar AUC values as those of the intermediate release composition. Since undesirable side effects are known to be triggered by steep increase of blood concentration, the higher Tₘₐₓ value can diminish side effect issues.

Provided herein in another aspect is a pharmaceutical composition comprising extended-release pellets and optionally delayed release pellets, wherein:
each of the extended-release pellets and each of the delayed release pellets comprises a drug pellet and an outer coating;
the outer coating of the extended-release pellets is an extended-release outer coating;
the outer coating of the delayed release pellets is a delayed release outer coating;
the drug pellet comprises about 15 wt. % to about 25 wt. % ibudilast, about 60 wt. % to about 70 wt. % sugar sphere, about 5 wt. % to about 10 wt. % hydroxypropyl methylcellulose, and about 4 wt. % to about 8 wt. % silicon dioxide;
the extended-release outer coating comprises about 20 wt. % to about 55 wt. % ethyl cellulose, about 20 wt. % to about 55 wt. % hydroxypropyl methylcellulose, about 1 wt. % to about 4 wt. % triethyl citrate, and about 20 wt. % to about 25 wt. % talc, wherein the weight percentages of ethyl cellulose, hydroxypropyl methylcellulose, triethyl citrate, and talc are based on total weight of the extended-release outer coating; and
the delayed release outer coating comprises about 55 wt. % to about 65 wt. % copolymer of methacrylic acid and ethyl acrylate, about 3 wt. % to about 9 wt. % triethyl citrate, and about 25 wt. % to about 35 wt. % talc, wherein the weight percentages of the copolymer of methacrylic acid and ethyl acrylate, triethyl citrate, and talc are based on total weight of the delayed release outer coating.

In some embodiments, ethyl cellulose and hydroxypropyl methylcellulose are present in the extended-release outer coating in a ratio of wt. % ethyl cellulose to wt. % hydroxypropyl methylcellulose of 2:1 to 1:2. In some embodiments, ethyl cellulose and triethyl citrate are present in the extended-release outer coating in a ratio of wt. % ethyl cellulose to wt. % triethyl citrate of 100:7. In some embodiments, the drug pellet comprises about 22 wt. % ibudilast, about 65 wt. % sugar sphere, about 7 wt. % hydroxypropyl methylcellulose, and about 6 wt. % silicon dioxide, wherein the weight percentages of ibudilast, sugar sphere, hydroxypropyl methylcellulose, and silicon dioxide are based on total weight of the drug pellet. In some embodiments, the drug pellet is present in the extended-release pellet in an amount of about 90 wt. % to about 97 wt. %, and the extended-release outer coating is present in the extended-release pellet in an amount of about 3 wt. % to about 10 wt. %. In some embodiments, the drug pellet is present in the extended-release pellet in an amount of about 90.9 wt. %, and the extended-release outer coating is present in the extended-release pellet in an amount of about 9.1 wt. %. In some embodiments, the drug pellet is present in the extended-release pellet in an amount of about 93.0 wt. %, and the extended-release outer coating is present in the extended-release pellet in an amount of about 7.0 wt. %. In some embodiments, the drug pellet is present in the extended-release pellet in an amount of about 94.0 wt. %, and the extended-release outer coating is present in the extended-release pellet in an amount of about 6.0 wt. %. In some embodiments, the drug pellet is present in the extended-release pellet in an amount of about 94.3 wt. %, and the extended-release outer coating is present in the extended-release pellet in an amount of about 5.7 wt. %. In some embodiments, the drug pellet is present in the extended-release pellet in an amount of about 94.6 wt. %, and the extended-release outer coating is present in the extended-release pellet in an amount of about 5.4 wt. %. In some embodiments, the extended-release outer coating comprises about 37.4 wt. % ethyl cellulose, about 37.4 wt. % hydroxypropyl methylcellulose, about 2.6 wt. % triethyl citrate, and about 22.5 wt. % talc, wherein the weight percentages of ethyl cellulose, hydroxypropyl methylcellulose, triethyl citrate, and talc are based on total weight of the extended-release outer coating. In some embodiments, the extended-release outer coating comprises about 49.5 wt. % ethyl cellulose, about 24.8 wt. % hydroxypropyl methylcellulose, about 3.5 wt. % triethyl citrate, and about 22.2 wt. % talc, wherein the weight percentages of ethyl cellulose, hydroxypropyl methylcellulose, triethyl citrate, and talc are based on total weight of the extended-release outer coating. In some embodiments, the extended-release outer coating comprises about 25.3 wt. % ethyl cellulose, about 50.3 wt. % hydroxypropyl methylcellulose, about 1.7 wt. % triethyl citrate, and about 22.7 wt. % talc, wherein the weight percentages of ethyl cellulose, hydroxypropyl methylcellulose, triethyl citrate, and talc are based on total weight of the extended-release outer coating. In some embodiments, the pharmaceutical composition comprises no delayed release pellets. In some embodiments, the pharmaceutical composition comprises extended-release pellets and delayed release pellets. In some embodiments, the delayed release outer coating comprises about 62.4 wt. % of the copolymer of methacrylic acid and ethyl acrylate, 6.3 wt. % of triethyl citrate, and 31.3 wt. % talc, wherein the weight percentages of the copolymer of methacrylic acid and ethyl acrylate, triethyl citrate, and talc are based on total weight of the delayed release outer coating. In some embodiments, the drug pellet is present in the delayed release pellet in an amount of about 75 wt. % to about 90 wt. %, and the delayed release outer coating is present in the delayed release pellet in an amount of about 10 wt. % to about 25 wt. %. In some embodiments, the drug pellet is present in the delayed release pellet in an amount of about 80 wt. %, and the delayed release outer coating is present in the delayed release pellet in an amount of about 20 wt. %. In some embodiments, the drug pellet is present in the delayed release pellet in an amount of about 87 wt. %, and the delayed release outer coating is present in the delayed release pellet in an amount of about 13 wt. %. In some embodiments, the extended-release pellets and delayed release pellets are present in the pharmaceutical composition in a ratio of number of extended-release pellets to number of delayed release pellets of 95:5 to 30:70. In some embodiments, the extended-release pellets and delayed release pellets are present in the pharmaceutical composition in a ratio of number of extended-release pellets to number of delayed release pellets of about 80:20. In some embodiments, the extended-release pellets and delayed release pellets are present in the pharmaceutical composition in a ratio of number of extended-release pellets to number of delayed release pellets of about 50:50. In some embodiments, the pharmaceutical composition has a pharmacokinetic profile having a higher Tₘₐₓ value compared to a Tₘₐₓ value of an intermediate release formulation. In some embodiments, the pharmaceutical composition has a pharmacokinetic profile having a similar AUC value compared to AUC value of the intermediate release formulation. In some embodiments, the pharmaceutical composition is in the form of a capsule. In some embodiments, the capsule has about 10 mg to about 200 mg of ibudilast. In some embodiments, the capsule has about 50 mg of ibudilast. In some embodiments, ibudilast is the sole active agent.

Provided herein in another aspect is a method of treating a neurodegenerative disease or disorder in a subject in need thereof, the method comprising administering to the subject a therapeutically effective amount of a pharmaceutical composition disclosed herein. In some embodiments, the neurodegenerative disease or disorder is Alzheimer's disease, Senile dementia of the Alzheimer type, Pick's disease (lobar atrophy), syndromes combining progressive dementia with other prominent neurologic abnormalities, Huntington's disease, multiple system atrophy combining dementia with ataxia and/or manifestation of Parkinson's disease, progressive supranuclear palsy (Steele-Richardson-Olszewski), diffuse Lewy body disease, corticodentatinigral degeneration, Hallervorden-Spatz disease, progressive familial myoclonic epilepsy, symptoms of gradually developing abnormalities of posture and movement, paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, Gilles de la Tourette syndrome, progressive ataxia, cerebellar degenerations, spinocerebellar degenerations, cerebellar cortical degeneration, olivopontocerebellar atrophy (OPCA), spinocerebellar degenerations (Friedreich's ataxia and related disorders), central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), other forms of familial spinal muscular atrophy, primary lateral sclerosis, hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, syndromes of progressive visual loss, pigmentary degeneration of the retina (retinitis pigmentosa), hereditary optic atrophy (Leber's disease), Parkinson's disease and other extrapyramidal disorders, progressive supranuclear palsy (Steele-Richardson-Olszewski syndrome), torsion dystonia (torsion spasm, dystonia musculorum deformans), focal dystonias, motor neuron disease, progressive ataxias, multifocal motor neuropathy with conduction block, motor neuropathy with paraprocinemia, motor-predominant peripheral neuropathies, olivopontocerebellar atrophy, Azorean (Machado-Joseph) disease, familial progressive neurodegenerative diseases, familial amyotrophic lateral sclerosis, spinal muscular atrophies, familial spastic paraparesis, hereditary biochemical disorders, arthrogryposis muliplex congenital, or progressive juvenile bulbar palsy (Fazio-Londe), infantile (Werdnig-Hoffman disease), childhood onset, or adolescent (Wohlfart-Kugelberg-Welander disease), degenerative cervical myelopathy, familial HTLV-1 myelopathy, isolated FSP, or complicated FSP, superoxide dismutase deficiency, hexosaminidase A and B deficiency, androgen receptor mutation (Kennedy's syndrome), viral and prion diseases, myelopathy, progressive multifocal leukoencephalopathy, Creutzfeldt-Jakob disease, Gerstmann-Straussler-Scheinker disease, kuru, fatal familial insomnia, Alper's disease, primary progressive or secondary progressive multiple sclerosis, frontotemporal dementia, Wilson's disease, progressive neuropathic pain, ischemia caused by stroke, traumatic brain injury, or spinal cord injury.

Provided herein in another aspect is a method of treating cancer in a subject in need thereof, the method comprising administering to the subject a therapeutically effective amount of a pharmaceutical composition disclosed herein.

Provided herein in another aspect is a method of preventing metastasis of a cancer in a subject in need thereof, the method comprising administering to the subject a therapeutically effective amount of a pharmaceutical composition disclosed herein.

Provided herein in another aspect is a method of ameliorating metastasis of a cancer in a subject in need thereof, the method comprising administering to the subject a therapeutically effective amount of a pharmaceutical composition disclosed herein.

Provided herein in another aspect is a method of minimizing metastasis of a cancer in a subject in need thereof, the method comprising administering to the subject a therapeutically effective amount of a pharmaceutical composition disclosed herein.

Provided herein in another aspect is a method of preventing relapse of a cancer in a subject in need thereof, the method comprising administering to the subject a therapeutically effective amount of a pharmaceutical composition disclosed herein.

Provided herein in another aspect is a method of ameliorating relapse of a cancer in a subject in need thereof, the method comprising administering to the subject a therapeutically effective amount of a pharmaceutical composition disclosed herein.

Provided herein in another aspect is a method of minimizing risk of relapse or delaying relapse of a cancer in a subject in need thereof, the method comprising administering to the subject a therapeutically effective amount of a pharmaceutical composition disclosed herein.

In some embodiments, the cancer is
a. a cancer of the circulatory system selected from angiosarcoma, fibrosarcoma, rhabdomyosarcoma, liposarcoma, myxoma, rhabdomyoma, fibroma, lipoma and teratoma, cancer of the mediastinum and pleura, and a vascular tumor;
b. a cancer of the respiratory tract selected from cancer of the nasal cavity and middle ear, cancer of accessory sinuses, cancer of larynx, cancer of the trachea, cancer of the bronchus and lung, small cell lung cancer (SCLC), non-small cell lung cancer (NSCLC), bronchogenic carcinoma, squamous cell carcinoma, undifferentiated small cell carcinoma, undifferentiated large cell carcinoma, adenocarcinoma, alveolar (bronchiolar) carcinoma, bronchial adenoma, sarcoma, lymphoma, chondromatous hamartoma and mesothelioma;
c. a cancer of the gastrointestinal system selected from squamous cell carcinoma, adenocarcinoma, leiomyosarcoma, lymphoma, carcinoma, ductal adenocarcinoma, insulinoma, glucagonoma, gastrinoma, carcinoid tumors, vipoma, adenocarcinoma, carcinoid tumors, Karposi's sarcoma, hemangioma, lipoma, neurofibroma, fibroma, tubular adenoma, villous adenoma, hamartoma, and leiomyoma;
d. a cancer of the genitourinary tract selected from adenocarcinoma, Wilm's tumor (nephroblastoma), lymphoma, leukemia, squamous cell carcinoma, transitional cell carcinoma, adenocarcinoma, adenocarcinoma, sarcoma of the prostate, seminoma, teratoma, embryonal carcinoma, teratocarcinoma, choriocarcinoma, interstitial cell carcinoma, fibroma, fibroadenoma, adenomatoid tumors, and lipoma;
e. a cancer of the hepatobiliary and pancreatic system selected from hepatoma (hepatocellular carcinoma), cholangiocarcinoma, hepatoblastoma, angiosarcoma, hepatocellular adenoma, hemangioma, pheochromocytoma, insulinoma, vasoactive intestinal peptide tumor, islet cell tumor, pancreatic exocrine tumor, and pancreatic neuroendocrine tumor;
f. a cancer of the bone selected from osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors;
g. a cancer of the nervous system selected from primary CNS lymphoma, osteoma, hemangioma, granuloma, xanthoma, osteitis deformans, meningioma, meningiosarcoma, gliomatosis, astrocytoma, medulloblastoma, glioma, ependymoma, germinoma (pinealoma), oligodendroglioma, schwannoma, retinoblastoma, congenital tumors, spinal cord neurofibroma, meningioma, glioma, and sarcoma;
h. a cancer of the reproductive system selected from endometrial carcinoma, cervical carcinoma, pre-tumor cervical dysplasia, ovarian carcinoma, serous cystadenocarcinoma, mucinous cystadenocarcinoma, unclassified carcinoma, granulosa-thecal cell tumors, Sertoli-Leydig cell tumors, dysgerminoma, malignant teratoma, squamous cell carcinoma of the vulva, intraepithelial carcinoma of the vulva, adenocarcinoma of the vulva, fibrosarcoma of the vulva, melanoma of the vulva, vaginal clear cell carcinoma, vaginal squamous cell carcinoma, vaginal botryoid sarcoma (embryonal rhabdomyosarcoma), carcinoma of the fallopian tubes placental cancer, uterine cancer, penile cancer, prostate cancer, and testicular cancer;
i. cancer of the hematologic system selected from myeloid, acute lymphoblastic leukemia, chronic lymphocytic leukemia, myeloproliferative diseases, multiple myeloma, myelodysplastic syndrome, Hodgkin's disease, and non-Hodgkin's lymphoma;
j. a cancer of the oral cavity selected from lip cancer, tongue cancer, gum cancer, floor of mouth cancer, palate cancer, parotid gland cancer, salivary gland cancer, tonsil cancer, cancer of the oropharynx, cancer of the nasopharynx, pyriform sinus cancer, and cancer of the hypopharynx;
k. a cancer of the skin selected from malignant melanoma, cutaneous melanoma, basal cell carcinoma, squamous cell carcinoma, Karposi's sarcoma, moles dysplastic nevi, lipoma, angioma, dermatofibroma, and keloidal cancer; or
l. a cancer selected from cancer of the adrenal glands, neuroblastoma, cancer of connective and soft tissue, cancer of the retroperitoneum and peritoneum, eye cancer, intraocular melanoma, cancer of adnexa, breast cancer, head or/and neck cancer, anal cancer, thyroid cancer, parathyroid cancer, cancer of the adrenal gland, cancer of the endocrine glands and related structures, secondary and unspecified malignant neoplasm of lymph nodes, secondary malignant neoplasm of respiratory and digestive systems, and secondary malignant neoplasm of other sites.

Provided herein in another aspect is a method of treating an autoimmune disorder in a subject in need thereof, the method comprising administering to the subject a therapeutically effective amount of a pharmaceutical composition disclosed herein. In some embodiments, the autoimmune disorder rheumatoid arthritis, IgA nephropathy, vascular disease associated with kidney disease, systemic lupus erythematosus (SLE), Wegener's granulomatosis, relapsing polychondritis, atopic dermatitis, psoriasis, sarcoidosis, Behçet's disease, Vogt-Koyanagi-Harada's disease, uveitis, or idiopathic pulmonary fibrosis.

Provided herein in another aspect is a method of treating a microorganism infection in a subject in need thereof, the method comprising administering to the subject a therapeutically effective amount of a pharmaceutical composition disclosed herein. In some embodiments, the microorganism infection is caused by virus, bacteria, fungus, or any combination of two or more thereof.

Provided herein in another aspect is a method of treating sepsis and/or septic shock in a subject in need thereof, the method comprising administering to the subject a therapeutically effective amount of a pharmaceutical composition disclosed herein.

Provided herein in another aspect is a method of treating severe viral-induced pneumonia in a subject in need thereof, the method comprising administering to the subject a therapeutically effective amount of a pharmaceutical composition disclosed herein. In some embodiments, the severe viral-induced pneumonia is associated with an infection by a respiratory virus. In some embodiments, the respiratory virus is selected from an influenza virus, a respiratory syncytial virus, a coronavirus, a rhinovirus, an adenovirus, and a parainfluenza virus. In some embodiments, the coronavirus is COVID-19.

Provided herein in another aspect is a method of treating mild to severe acute respiratory distress syndrome (ARDS) in a subject in need thereof, the method comprising administering to the subject a therapeutically effective amount of a pharmaceutical composition disclosed herein, and wherein the subject has positive end-expiratory pressure (PEEP)≥5 cm H₂O; and PaO₂/FiO₂<300 mm Hg. In some embodiments, the subject has positive end-expiratory pressure (PEEP)≥5 cm H₂O; and PaO₂/FiO₂<200 mm Hg. In some embodiments, the ARDS is associated with an infection by a respiratory virus. In some embodiments, the respiratory virus is selected from an influenza virus, a respiratory syncytial virus, a coronavirus, a rhinovirus, an adenovirus, and a parainfluenza virus. In some embodiments, the coronavirus is COVID-19.

Provided herein in another aspect is a method of treating fragile X syndrome in a subject in need thereof, the method comprising administering to the subject a therapeutically effective amount of a pharmaceutical composition disclosed herein.

Provided herein in another aspect is a method of treating acute lung injury in a subject in need thereof, the method comprising administering to the subject a therapeutically effective amount of the pharmaceutical composition disclosed herein. In some embodiments, the lung injury is induced by aspiration, trauma, pancreatitis, blood transfusion, or smoke or toxic chemical inhalation. In some embodiments, the lung injury is induced by a chemical selected from chlorine, sulfur mustard gas, phosgene, Lewisite, hydrogen chloride, sulfur dioxide, hydrogen sulfide, nitrogen dioxide, ammonia, hydrofluoric acid, ozone, methyl isocyanate, and a combination of two or more thereof. In some embodiments, the lung injury comprises chemical burns, pulmonary edema, laryngeal edema, lung tissue apoptosis, pneumonia, pneumonitis, bronchitis, bronchiolitis, fibrosis, acute respiratory distress syndrome, respiratory tract spasm, or a combination of two or more thereof.

### DETAILED DESCRIPTION

The practice of the present disclosure will employ, unless otherwise indicated, conventional methods of chemistry, biochemistry, and pharmacology, within the skill of the art. Such techniques are explained fully in the literature. See, e.g., A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20th Ed.; FDA's Orange Book, Goodman & Gilman The Pharmacological Basis of Therapeutics, J. Griffith Hardman, L. L. Limbird, A. Gilman, 11th Ed., 2005, The Merck Manual, 18th edition, 2007, and The Merck Manual of Medical Information 2003.

All publications cited herein, including internet articles, the FDA Orange Book (available on the FDA's website), books, handbooks, journal articles, patents and patent applications, whether supra or infra, are hereby incorporated by reference in their entirety.

### Definitions

Before describing the present disclosure in detail, it is to be understood that this disclosure is not limited to particular administration modes, patient populations, and the like, as such may vary, as will be apparent from the accompanying description and figures.

It must be noted that, as used in this specification and the intended claims, the singular forms “a,” “an,” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

In describing and claiming the present disclosure, the following terminology will be used in accordance with the definitions described below.

“Optional” or “optionally” means that the subsequently described circumstance may or may not occur, so that the description includes instances where the circumstance occurs and instances where it does not.

“Pharmaceutically acceptable excipient or carrier” refers to an excipient that may optionally be included in the compositions of the disclosure and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in vivo or in vitro. This includes foods, food supplements, nutrients, nutraceuticals, drugs, vaccines, antibodies, vitamins, and other beneficial agents. As used herein, the terms further include any physiologically or pharmacologically active substance that produces a localized or systemic effect in a patient. In specific embodiments, the active molecule or active agent may include ibudilast or a pharmaceutically acceptable salt thereof.

“Substantially” or “essentially” means nearly totally or completely, for instance, 90% or 95% or greater of some given quantity.

The terms “subject,” “individual” or “patient” are used interchangeably herein and refer to a vertebrate, preferably a mammal. Mammals include, but are not limited to, mice, rodents, rats, simians, humans, farm animals, dogs, cats, sport animals and pets. In some embodiments, subject, individual, or patient is in reference to a human.

The terms “pharmacologically effective amount” or “therapeutically effective amount” of a composition or agent, as provided herein, refer to a nontoxic but sufficient amount of the composition or agent to provide the desired response, such as a reduction or reversal of any disease or disorder described herein. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

As used herein, the term “sugar sphere” (also known as sugar bead) refers to a neutral pellet or nonpareil seed of spherical shape consisting of sucrose and cornstarch. In some embodiments, the sugar sphere has a diameter of about 700 μm to about 900 μm, or about 710 μm to about 850 μm.

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

As used herein, the term “treatment” or “treating” means any treatment of a disease or condition or associated disorder, in a patient, including inhibiting the disease or condition, that is, arresting or suppressing the development of clinical symptoms. “Treatment” or “treating” also includes arresting the development of or reversing the symptom or symptoms of a disease. For purposes of the various aspects and embodiments of the present disclosure, beneficial or desired clinical results include, but are not limited to, reduction, alleviation, or amelioration of one or more manifestations of or negative effects of any disease or disorder described herein, improvement in one or more clinical outcomes, diminishment of extent of any disease or disorder described herein, delay or slowing of any disease or disorder described herein progression, amelioration, palliation, or stabilization of the any disease or disorder described herein state, and other beneficial results described herein.

In some aspects, the term “treating” refers to an improvement in clinical outcomes. The term “clinical outcome” refers to any clinical observation or measurement relating to a patient's reaction to a therapy.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Described herein are extended release formulations of ibudilast for oral administration.

### Ibudilast

The methods of the disclosure are based upon administration of the molecule, ibudilast. Ibudilast is a small molecule drug (molecular weight of 230.3) having the structure shown below.

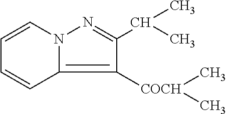

Ibudilast is also found under ChemBank ID 3227, CAS #50847-11-5, and Beilstein Handbook Reference No. May 24, 2003-00396. Its molecular formula corresponds to C₁₄H₁₈N₂O. Ibudilast is also known by various chemical names including 2-methyl-1-(2-(1-methylethyl) pyrazolo (1,5-a)pyridin-3-yl) 1-propanone; 3-isobutyryl-2-isopropylpyrazolo (1,5-a) pyridine; and 1-(2-isopropyl-pyrazolo[1,5-a]pyridin-3-yl)-2-methyl-propan-1-one. Other synonyms for ibudilast include Ibudilastum (Latin), BRN 0656579, KC-404, and MN-166. Its brand name is KETAS®. Ibudilast, as referred to herein, is meant to include any and all pharmaceutically acceptable salt forms thereof, prodrug forms (e.g., the corresponding ketal), solvates, and the like, as appropriate for use in its intended formulation for administration.

Ibudilast is an inhibitor of the macrophage inhibitory factor (MIF). Ibudilast is also a selective inhibitor of cyclic nucleotide phosphodiesterases (PDEs) 3A, 4, 10A1 and 11A1 (Gibson et al., Eur. J. Pharmacol. 538:39-42, 2006), and has also been reported to have leukotriene D4 and PAF antagonistic activities. Its profile appears effectively anti-inflammatory and unique in comparison to other PDE inhibitors and anti-inflammatory agents. PDEs catalyze the hydrolysis of the phosphoester bond on the 3′-carbon to yield the corresponding 5′-nucleotide monophosphate. Thus, they regulate the cellular concentrations of cyclic nucleotides. Since extracellular receptors for many hormones and neurotransmitters utilize cyclic nucleotides as second messengers, the PDEs also regulate cellular responses to these extracellular signals. There are at least eight classes of PDEs: Ca²⁺/calmodulin-dependent PDEs (PDE1); cGMP-stimulated PDEs (PDE2); cGMP-inhibited PDEs (PDE3); cAMP-specific PDEs (PDE4); cGMP-binding PDEs (PDE5); photoreceptor PDEs (PDE6); high affinity, cAMP-specific PDEs (PDE7); and high affinity cGMP-specific PDEs (PDE9). Ibudilast acts to suppress inflammation via action on inflammatory cells (e.g., glial cells) resulting in the suppression of both pro-inflammatory mediator and neuroactive mediator release. Ibudilast may also suppress the production of pro-inflammatory cytokines (IL-1ß, TNF-α) and may enhance the production of the anti-inflammatory cytokines (IL-4, IL-10). References related to the foregoing include the following: Obernolte, R., et al. (1993) “The cDNA of a human lymphocyte cyclic-AMP phosphodiesterase (PDE IV) reveals a multigene family” Gene 129:239-247; Rile, G., et al. (2001) “Potentiation of ibudilast inhibition of platelet aggregation in the presence of endothelial cells” Thromb. Res. 102:239-246; Souness, J. E., et al. (1994) “Possible role of cyclic AMP phosphodiesterases in the actions of ibudilast on eosinophil thromboxane generation and airways smooth muscle tone” Br. J. Pharmacol. 111:1081-1088; Suzumura, A., et al. (1999) “Ibudilast suppresses TNF-alpha production by glial cells functioning mainly as type III phosphodiesterase inhibitor in CNS” Brain Res. 837:203-212; Takuma, K., et al. (2001) “Ibudilast attenuates astrocyte apoptosis via cyclic GMP signaling pathway in an in vitro reperfusion model” Br. J. Pharmacol. 133:841-848. Ibudilast exhibits good CNS penetration; Sanftner et al. Xenobiotica, (2009) 39:964-977.

As stated previously, a reference to any one or more of the herein-described drugs, in particular ibudilast, is meant to encompass, where applicable, any and all enantiomers, mixtures of enantiomers including racemic mixtures, prodrugs, pharmaceutically acceptable salt forms, hydrates (e.g., monohydrates, dihydrates, etc.), solvates, different physical forms (e.g., crystalline solids, amorphous solids), metabolites, and the like.

### Pharmaceutical Compositions

In one aspect, described herein are pharmaceutical compositions comprising, consisting essentially of, or consisting of extended-release pellets and optionally delayed release pellets, wherein:
each of the extended-release pellets and each of the delayed release pellets comprises, consists essentially of, or consists of a drug pellet and an outer coating;
the outer coating of the extended-release pellets is an extended-release outer coating;
the outer coating of the delayed release pellets is a delayed release outer coating;
the drug pellet comprises, consists essentially of, or consists of about 15 wt. % to about 25 wt. % ibudilast, about 60 wt. % to about 70 wt. % sugar sphere, about 5 wt. % to about 10 wt. % hydroxypropyl methylcellulose, and about 4 wt. % to about 8 wt. % silicon dioxide;
the extended-release outer coating comprises, consists essentially of, or consists of about 20 wt. % to about 55 wt. % ethyl cellulose, about 20 wt. % to about 55 wt. % hydroxypropyl methylcellulose, about 1 wt. % to about 4 wt. % triethyl citrate, and about 20 wt. % to about 25 wt. % talc, wherein the weight percentages of ethyl cellulose, hydroxypropyl methylcellulose, triethyl citrate, and talc are based on total weight of the extended-release outer coating; and
the delayed release outer coating comprises, consists essentially of, or consists of about 55 wt. % to about 65 wt. % copolymer of methacrylic acid and ethyl acrylate, about 3 wt. % to about 9 wt. % triethyl citrate, and about 25 wt. % to about 35 wt. % talc, wherein the weight percentages of the copolymer of methacrylic acid and ethyl acrylate, triethyl citrate, and talc are based on total weight of the delayed release outer coating.

A sugar sphere is present in the drug pellet in an amount of about 60 wt. % to about 70 wt. %. This includes an amount of about 60 wt. %, about 61 wt. %, about 62 wt. %, about 63 wt. %, about 64 wt. %, about 65 wt. %, about 66 wt. %, about 67 wt. %, about 68 wt. %, about 69 wt. %, or about 70 wt. %, or any value therebetween. In some embodiments, a sugar sphere is present in the drug pellet in an amount of about 65 wt. % to about 66 wt. %. In some embodiments, a sugar sphere is present in the drug pellet in an amount of about 65 wt. %. In some embodiments, a sugar sphere is present in the drug pellet in an amount of about 65.1 wt. %.

Hydroxypropyl methylcellulose (HPMC), also known as hypromellose (such as, but not limited to, METHOCEL™ E5 Premium LV), is present in the drug pellet in an amount of about 5 wt. % to about 10 wt. %. This includes an amount of about 5 wt. %, about 6 wt. %, about 7 wt. %, about 8 wt. %, about 9 wt. %, or about 10 wt. %, or any value therebetween. In some embodiments, hydroxypropyl methylcellulose is present in the drug pellet in an amount of about 7 wt. % to about 8 wt. %. In some embodiments, hydroxypropyl methylcellulose is present in the drug pellet in an amount of about 7 wt. %. In some embodiments, hydroxypropyl methylcellulose is present in the drug pellet in an amount of about 7.4 wt. %.

Silicon dioxide (such as, but not limited to, SYLOID® 244 FP silica) is present in the drug pellet in an amount of about 4 wt. % to about 8 wt. %. This includes an amount of about 4 wt. %, about 5 wt. %, about 6 wt. %, about 7 wt. %, or about 8 wt. %, or any value therebetween. In some embodiments, silicon dioxide is present in the drug pellet in an amount of about 5 wt. % to about 6 wt. %. In some embodiments, silicon dioxide is present in the drug pellet in an amount of about 6 wt. %. In some embodiments, silicon dioxide is present in the drug pellet in an amount of about 5.7 wt. %.

Ibudilast is present in the drug pellet in an amount of about 15 wt. % to about 25 wt. %. This includes an amount of about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, or about 25 wt. %, or any value therebetween. In some embodiments, ibudilast is present in the drug pellet in an amount of about 21 wt. % to about 22 wt. %. In some embodiments, ibudilast is present in the drug pellet in an amount of about 22 wt. %. In some embodiments, ibudilast is present in the drug pellet in an amount of about 21.9 wt. %.

In some embodiments, the drug pellet comprises, consists essentially of, or consists of about 22 wt. % ibudilast, about 65 wt. % sugar sphere, about 7 wt. % hydroxypropyl methylcellulose, and about 6 wt. % silicon dioxide, wherein the weight percentages of ibudilast, sugar sphere, hydroxypropyl methylcellulose, and silicon dioxide are based on total weight of the drug pellet.

Ethyl cellulose (such as, but not limited to, ETHOCEL™ Standard 7 Premium ethyl cellulose) is present in the extended-release outer coating in an amount of about 20 wt. % to about 55 wt. %. This includes about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 wt. %, or any value therebetween. In some embodiments, ethyl cellulose is present in the extended-release outer coating in an amount of about 37 wt. % to about 38 wt. %. In some embodiments, ethyl cellulose is present in the extended-release outer coating in an amount of about 37.4 wt. %. In some embodiments, ethyl cellulose is present in the extended-release outer coating in an amount of about 25 wt. % to about 26 wt. %. In some embodiments, ethyl cellulose is present in the extended-release outer coating in an amount of about 25.3 wt. %. In some embodiments, ethyl cellulose is present in the extended-release outer coating in an amount of about 49 wt. % to about 50 wt. %. In some embodiments, ethyl cellulose is present in the extended-release outer coating in an amount of about 49.5 wt. %.

Hydroxypropyl methylcellulose (such as, but not limited to, METHOCEL™ E5 Premium LV) is present in the extended-release outer coating in an amount of about 20 wt. % to about 55 wt. %. This includes about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 wt. %, or any value therebetween. In some embodiments, hydroxypropyl methylcellulose is present in the extended-release outer coating in an amount of about 37 wt. % to about 38 wt. %. In some embodiments, hydroxypropyl methylcellulose is present in the extended-release outer coating in an amount of about 37.4 wt. %. In some embodiments, hydroxypropyl methylcellulose is present in the extended-release outer coating in an amount of about 50 wt. % to about 51 wt. %. In some embodiments, hydroxypropyl methylcellulose is present in the extended-release outer coating in an amount of about 50.3 wt. %. In some embodiments, hydroxypropyl methylcellulose is present in the extended-release outer coating in an amount of about 24 wt. % to about 25 wt. %. In some embodiments, hydroxypropyl methylcellulose is present in the extended-release outer coating in an amount of about 24.8 wt. %.

Triethyl citrate is present in the extended-release outer coating in an amount of about 1 wt. % to about 4 wt. %. This includes about 1, 2, 3, or 4 wt. %, or any value therebetween. In some embodiments, triethyl citrate is present in the extended-release outer coating in an amount of about 2 wt. % to about 3 wt. %. In some embodiments, triethyl citrate is present in the extended-release outer coating in an amount of about 2.6 wt. %. In some embodiments, triethyl citrate is present in the extended-release outer coating in an amount of about 3 wt. % to about 4 wt. %. In some embodiments, triethyl citrate is present in the extended-release outer coating in an amount of about 3.5 wt. %. In some embodiments, triethyl citrate is present in the extended-release outer coating in an amount of about 1 wt. % to about 2 wt. %. In some embodiments, triethyl citrate is present in the extended-release outer coating in an amount of about 1.7 wt. %.

Talc is present in the extended-release outer coating in an amount of about 20 wt. % to about 25 wt. %. This includes about 20, 21, 22, 23, 24, or 25 wt. %, or any value therebetween. In some embodiments, talc is present in the extended-release outer coating in an amount of about 22 wt. % to about 23 wt. %. In some embodiments, talc is present in the extended-release outer coating in an amount of about 22.5 wt. %. In some embodiments, talc is present in the extended-release outer coating in an amount of about 22.2 wt. %. In some embodiments, talc is present in the extended-release outer coating in an amount of about 22.7 wt. %. In some embodiments, the talc is ultra micronized.

In some embodiments, ethyl cellulose and hydroxypropyl methylcellulose are present in the extended-release outer coating in a ratio of wt. % ethyl cellulose to wt. % hydroxypropyl methylcellulose of 2:1 to 1:2. This includes 2:1, 1:1, or 1:2, or any value therebetween.

In some embodiments, ethyl cellulose and triethyl citrate are present in the extended-release outer coating in a ratio of wt. % ethyl cellulose to wt. % triethyl citrate of 100:7.

In some embodiments, talc is present in the extended-release outer coating in an amount of about 30 wt. % relative to total weight percent of ethyl cellulose and hydroxypropyl methylcellulose in the extended-release outer coating. In a non-limiting example of such embodiments, ethyl cellulose and hydroxypropyl methylcellulose are each present in the extended-release outer coating in an amount of about 37.44 wt. %, thereby accounting for about 74.88 wt. % in the extended-release outer coating, and talc is present in an amount of about 22.5 wt. %.

In some embodiments, the extended-release outer coating comprises, consists essentially of, or consists of about 37.4 wt. % ethyl cellulose, about 37.4 wt. % hydroxypropyl methylcellulose, about 2.6 wt. % triethyl citrate, and about 22.5 wt. % talc, wherein the weight percentages of ethyl cellulose, hydroxypropyl methylcellulose, triethyl citrate, and talc are based on total weight of the extended-release outer coating.

In some embodiments, the extended-release outer coating comprises, consists essentially of, or consists of about 49.5 wt. % ethyl cellulose, about 24.8 wt. % hydroxypropyl methylcellulose, about 3.5 wt. % triethyl citrate, and about 22.2 wt. % talc, wherein the weight percentages of ethyl cellulose, hydroxypropyl methylcellulose, triethyl citrate, and talc are based on total weight of the extended-release outer coating.

In some embodiments, the extended-release outer coating comprises, consists essentially of, or consists of about 25.3 wt. % ethyl cellulose, about 50.3 wt. % hydroxypropyl methylcellulose, about 1.7 wt. % triethyl citrate, and about 22.7 wt. % talc, wherein the weight percentages of ethyl cellulose, hydroxypropyl methylcellulose, triethyl citrate, and talc are based on total weight of the extended-release outer coating.

In some embodiments, the drug pellet is present in the extended-release pellet in an amount of about 90 wt. % to about 97 wt. %, and the extended-release outer coating is present in the extended-release pellet in an amount of about 3 wt. % to about 10 wt. %. This includes the drug pellet being present in the extended-release pellet in an amount of about 90, 91, 92, 93, 94, 95, 96, or 97 wt. %, or any value therebetween, and the extended-release outer coating being present in the extended-release pellet in an amount of about 3, 4, 5, 6, 7, 8, 9, or 10 wt. %, or any value therebetween. In some embodiments, the drug pellet is present in the extended-release pellet in an amount of about 90.9 wt. %, and the extended-release outer coating is present in the extended-release pellet in an amount of about 9.1 wt. %. In some embodiments, the drug pellet is present in the extended-release pellet in an amount of about 93.0 wt. %, and the extended-release outer coating is present in the extended-release pellet in an amount of about 7.0 wt. %. In some embodiments, the drug pellet is present in the extended-release pellet in an amount of about 94.0 wt. %, and the extended-release outer coating is present in the extended-release pellet in an amount of about 6.0 wt. %. In some embodiments, the drug pellet is present in the extended-release pellet in an amount of about 94.3 wt. %, and the extended-release outer coating is present in the extended-release pellet in an amount of about 5.7 wt. %. In some embodiments, the drug pellet is present in the extended-release pellet in an amount of about 94.6 wt. %, and the extended-release outer coating is present in the extended-release pellet in an amount of about 5.4 wt. %.

In some embodiments, the pharmaceutical composition comprises, consists essentially of, or consists of extended-release pellets. In some embodiments, the pharmaceutical composition comprises no delayed release pellets. In some embodiments, the pharmaceutical composition comprises, consists essentially of, or consists of extended-release pellets and delayed release pellets.

A copolymer of methacrylic acid and ethyl acrylate (such as, but not limited to EUDRAGIT® L30 D55) is present in the delayed release outer coating in an amount of about 55 wt. % to about 65 wt. %. This includes about 55, 56, 57, 58, 59, 60, 61, 62, 63, 64, or 65 wt. %, or any value therebetween. In some embodiments, the copolymer of methacrylic acid and ethyl acrylate is present in the delayed release outer coating in an amount of about 62 wt. % to about 63 wt. %. In some embodiments, the copolymer of methacrylic acid and ethyl acrylate is present in the delayed release outer coating in an amount of about 62.4 wt. %.

Triethyl citrate is present in the delayed release outer coating in an amount of about 3 wt. % to about 9 wt. %. This includes about 3, 4, 5, 6, 7, 8, or 9 wt. %, or any value therebetween. In some embodiments, triethyl citrate is present in the delayed release outer coating in an amount of about 6 wt. % to about 7 wt. %. In some embodiments, triethyl citrate is present in the delayed release outer coating in an amount of about 6.3 wt. %.

Talc is present in the delayed release outer coating in an amount of about 25 wt. % to about 35 wt. %. This includes about 25, 26, 27, 28, 29, 30, 31, 32, 33, 34, or 35 wt. %, or any value therebetween. In some embodiments, talc is present in the delayed release outer coating in an amount of about 31 wt. % to about 32 wt. %. In some embodiments, talc is present in the delayed release outer coating in an amount of about 31.3 wt. %.

In some embodiments, the delayed release outer coating comprises, consists essentially of, or consists of about 62.4 wt. % of the copolymer of methacrylic acid and ethyl acrylate, 6.3 wt. % of triethyl citrate, and 31.3 wt. % talc, wherein the weight percentages of the copolymer of methacrylic acid and ethyl acrylate, triethyl citrate, and talc are based on total weight of the delayed release outer coating.

In some embodiments, the drug pellet is present in the delayed release pellet in an amount of about 75 wt. % to about 90 wt. %, and the delayed release outer coating is present in the delayed release pellet in an amount of about 10 wt. % to about 25 wt. %. This includes the drug pellet being present in the delayed release pellet in an amount of about 75, 76, 77, 78, 79, 80, 81, 82, 83, 84, 85, 86, 87, 88, 89, or 90 wt. %, or any value therebetween, and the delayed release outer coating being present in the delayed release pellet in an amount of about 10, 11, 12, 13, 14, 15, 16, 17, 18, 19, 20, 21, 22, 23, 24, or 25 wt. %, or any value therebetween. In some embodiments, the drug pellet is present in the delayed release pellet in an amount of about 80 wt. %, and the delayed release outer coating is present in the delayed release pellet in an amount of about 20 wt. %. In some embodiments, the drug pellet is present in the delayed release pellet in an amount of about 87 wt. %, and the delayed release outer coating is present in the delayed release pellet in an amount of about 13 wt. %.

In some embodiments, the extended-release pellets and delayed release pellets are present in the pharmaceutical composition in a ratio of number of extended-release pellets to number of delayed release pellets of 95:5 to 30:70. This includes a ratio of number of extended-release pellets to number of delayed release pellets of 95:5, 90:10, 85:15, 80:20, 75:25, 70:30, 65:35, 60:40, 55:45, 50:50, 45:55, 40:60, 35:65, or 30:70, including any value therebetween. In some embodiments, the extended-release pellets and delayed release pellets are present in the pharmaceutical composition in a ratio of number of extended-release pellets to number of delayed release pellets of about 90:10. In some embodiments, the extended-release pellets and delayed release pellets are present in the pharmaceutical composition in a ratio of number of extended-release pellets to number of delayed release pellets of about 80:20. In some embodiments, the extended-release pellets and delayed release pellets are present in the pharmaceutical composition in a ratio of number of extended-release pellets to number of delayed release pellets of about 50:50.

In some embodiments, the pharmaceutical composition is in the form of a capsule. In other words, the extended release pellets and, if present, the delayed release pellets are comprised within a capsule. In some embodiments, the capsule has about 10 mg to about 200 mg ibudilast. This includes about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg, including any value therebetween. In some embodiments, the capsule has about 50 mg ibudilast.

In some embodiments, the extended release pellets are spherical. In some embodiments, the delayed release pellets are spherical. In some embodiments, the extended release pellets and the delayed release pellets are spherical. In some embodiments, the extended release pellets and the delayed release pellets are spherical and have a diameter of about 700 microns to about 1200 microns. This includes about 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, or 1200 microns, or any value therebetween. In some embodiments, the extended release pellets and the delayed release pellets are similar in diameter. In some embodiments, the extended release pellets and the delayed release pellets do not have the same diameter.

In some embodiments, the pharmaceutical composition described herein has a pharmacokinetic profile having a higher Tₘₐₓ value compared to a Tₘₐₓ value of an intermediate release formulation. For example, the pharmaceutical composition described herein has a pharmacokinetic profile having a higher Tₘₐₓ value under fed conditions compared to a Tₘₐₓ value of an intermediate release formulation under fasted conditions. In some embodiments, the pharmaceutical composition described herein has a pharmacokinetic profile having a similar AUC value compared to AUC value of an intermediate release formulation.

### Methods of Treatment

In another aspect, any one of the compositions described herein are used to treat any one or more of the diseases or disorders described herein. Accordingly, a method to treat one or more of the diseases or disorders described herein using a therapeutically effective amount of a composition described herein is another aspect of this disclosure.

In some embodiments, the disease or disorder is neurodegenerative disease, hereditary biochemical disorder, progressive neurodegenerative disease, or symptoms thereof. Exemplary neurodegenerative diseases/disorders include, but are not limited to, Alzheimer's disease, Senile dementia of the Alzheimer type, or Pick's disease (lobar atrophy), multiple sclerosis, neurodegenerative diseases that include syndromes combining progressive dementia with other prominent neurologic abnormalities, progressive neurodegenerative disease mainly afflicting adults and including progressive neurodegenerative forms of Huntington's disease, multiple system atrophy combining dementia with ataxia and/or manifestation of Parkinson's disease, progressive supranuclear palsy (Steele-Richardson-Olszewski), diffuse Lewy body disease, or corticodentatinigral degeneration. Additional subjects can be suffering from progressive neurodegenerative disease that mainly afflicts young adults and children and include Hallervorden-Spatz disease and progressive familial myoclonic epilepsy, progressive neurodegenerative disease that includes syndromes of gradually developing abnormalities of posture and movement, or disease that includes paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, or Gilles de la Tourette syndrome, syndromes of progressive ataxia, cerebellar degenerations or spinocerebellar degenerations, cerebellar cortical degeneration or olivopontocerebellar atrophy (OPCA), spinocerebellar degenerations including spinocerebellar degenerations (Friedreich's ataxia and related disorders). Neurodegenerative diseases/disorders include, but are not limited to, central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), or other forms of familial spinal muscular atrophy, primary lateral sclerosis or hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, progressive neurodegenerative diseases that include syndromes of progressive visual loss. Neurodegenerative diseases/disorders include, but are not limited to, pigmentary degeneration of the retina (retinitis pigmentosa), or hereditary optic atrophy (Leber's disease), motor neuron disease and the progressive ataxias; glaucoma; retinal detachment; sporadic progressive neurodegenerative diseases, multifocal motor neuropathy with conduction block, motor neuropathy with paraprocinemia, motor-predominant peripheral neuropathies, olivopontocerebellar atrophy, Azorean (Machado-Joseph) disease, familial progressive neurodegenerative diseases such as familial amyotrophic lateral sclerosis, spinal muscular atrophies, familial spastic paraparesis, hereditary biochemical disorders, arthrogryposis multiplex congenital, or progressive juvenile bulbar palsy (Fazio-Londe). Examples of hereditary biochemical disorders are superoxide dismutase deficiency, hexosaminidase A and B deficiency, or androgen receptor mutation (Kennedy's syndrome). Progressive neurodegenerative diseases can include viral and prion diseases, such as HTL V-1 associated myelopathy, progressive multifocal leukoencephalopathy, Creutzfeldt-Jakob disease, Gerstmann-Straussler-Scheinker disease, kuru, fatal familial insomnia, or Alper's disease.

“Progressive neurodegenerative disease” means any neurodegenerative disease that is in the progressive state (that is, getting worse compared to a baseline level) or has such progressive characteristics. Thus, a progressive state is a worsening of symptoms over time and can be precipitous or gradual. Examples of progressive neurodegenerative diseases include Parkinson's disease, amyotrophic lateral sclerosis, Alzheimer's disease, and progressive forms of multiple sclerosis exclusive of relapse/remitting multiple sclerosis.

In some embodiments, the disease or disorder is an ophthalmic disease/disorder or injury associated with a neurodegenerative disease/disorder or a neuro-ophthalmologic disorder. In some embodiments, the ophthalmic disease/disorder or injury is retinal injury. In some embodiments, the ophthalmic disease/disorder or injury is macular injury. In some embodiments, the ophthalmic disease/disorder or injury is macular thinness. In some embodiments, the neurodegenerative disease/disorder is progressive multiple sclerosis. Exemplary neuro-ophthalmologic disorders include, but are not limited to, papilledema and idiopathic intracranial hypertension (IIH); anterior ischemic optic neuropathy (AION); optic neuritis; ocular motor cranial neuropathy; and Horner syndrome.

In some embodiments, the disease or disorder is alcoholism and depression and/or dysphoric mood. Examples include alcohol use disorder (AUD) that may be accompanied with depression or dysphoric mood. In some embodiments, the depression is mild depression, moderate depression, or severe depression. In some embodiments, the dysphoric mood is higher dysphoric mood, moderate dysphoric mood, or lower dysphoric mood.

In some embodiments, the disease or disorder is glioblastoma, recurrent glioblastoma or its associated symptoms. In some embodiments, the disease or disorder is glioblastoma. In some embodiments, the disease or disorder is recurrent glioblastoma.

For treating glioblastoma or recurrent glioblastoma, in some embodiments, the patient is a human patient. In some embodiments, the patient has extra copies of the epidermal growth factor receptor (EGFR) gene or expresses abnormally high levels of EGFR. In some embodiments, the abnormally high levels of EGRF refers to higher levels of EGRF in a cancer patient relative to lower levels of EGFR in cancer-free individuals. In some embodiments, the patient lacks heterozygosity in chromosome 10. In some embodiments, the patient displays chromosome 7 amplification. In some embodiments, the patient has a mutated gene selected from the group consisting of TP53, PDGFRA, IDH1, PTEN and NF1. In some embodiments, the patient expresses NEFL, GABRA1, SYT1 or SLC12A5.

In some embodiments, the disease or disorder is multiple sclerosis or progressive multiple sclerosis. There are four recognized types of multiple sclerosis: (1) Relapsing/Remitting Multiple Sclerosis (RR multiple sclerosis), (2) Secondary Progressive Multiple Sclerosis (SP multiple sclerosis), (3) Progressive Relapsing Multiple Sclerosis (PR multiple sclerosis), and (4) Primary Progressive Multiple Sclerosis (PP multiple sclerosis). RR multiple sclerosis is not considered to fall within the scope of the claims, but the other forms of multiple sclerosis, i.e., SP multiple sclerosis, PR multiple sclerosis and PP multiple sclerosis are considered to be one aspect of the present invention. In all types of progressive MS, there is a loss of function over time regardless of relapses.

Relapsing/Remitting Multiple Sclerosis (RR multiple Sclerosis) is characterized by relapses (also known as exacerbations) during which time new symptoms can appear and old ones resurface or worsen. The relapses are followed by periods of remission, during which time the person fully or partially recovers from the deficits acquired during the relapse. Relapses can last for days, weeks or months and recovery can be slow and gradual or almost instantaneous. The vast majority of people presenting with Multiple Sclerosis are first diagnosed with relapsing/remitting. This is typically when they are in their twenties or thirties, though diagnoses much earlier or later are known. Around twice as many women as men present with this variety.

In Secondary Progressive Multiple Sclerosis (SP multiple Sclerosis), a person who initially had relapsing-remitting multiple Sclerosis begins to develop a gradual deterioration in nerve function, with or without relapses. After a number of years many people who have had relapsing/remitting multiple Sclerosis will pass into a secondary progressive phase of the disease. This is characterized by a gradual worsening of the disease between relapses. In the early phases of Secondary Progressive MS, the person may still experience a few relapses but after a while, these merge into a general progression. People often do not return to their prior level of function after a relapse. People with Secondary Progressive MS may experience good and bad days or weeks, but, apart from some remission following relapsing episodes, have no real recovery. After 10 years, 50% of people with relapsing/remitting multiple sclerosis will have developed secondary progressive. By 25 to 30 years, that figure will have risen to 90%.

Progressive Relapsing Multiple Sclerosis (PR multiple sclerosis) shows clear progression in the level of disability from the time symptoms first begin, but with episodes of clear relapses that may or may not be associated with some recovery following the acute episode. This form of multiple sclerosis follows a progressive course from onset, punctuated by relapses. There is significant recovery immediately following a relapse but between relapses, there is a gradual worsening of symptoms.

Primary Progressive Multiple Sclerosis (PP multiple sclerosis) is characterized by a gradual progression of the disease from its onset with no remissions or relapses at all. There may be periods of a leveling off of disease activity and, as with secondary progressive, there may be good and bad days or weeks. PP multiple sclerosis differs from Relapsing/Remitting MS and Secondary Progressive MS in that onset is typically in the late thirties or early forties, men are as likely women to develop it and initial disease activity is in the spinal cord and not in the brain. Primary Progressive multiple sclerosis often migrates into the brain, but is less likely to damage brain areas than relapsing/remitting or secondary progressive—for example, people with Primary Progressive MS are less likely to develop cognitive problems.

In some embodiments, the progressive multiple sclerosis has progressed beyond relapse remitting multiple sclerosis. In some embodiments, the progressive multiple sclerosis is primary progressive multiple sclerosis. In some embodiments, the primary progressive multiple sclerosis is characterized by disease progression from onset, with occasional plateaus and temporary minor improvements allowed, but not distinct relapses. In some embodiments, the progressive multiple sclerosis is secondary progressive multiple sclerosis. In some embodiments, the secondary progressive multiple sclerosis is characterized as an initial relapsing-remitting course, followed by progression, with or without occasional relapses, minor remissions and plateaus.

In some embodiments, the disease or disorder is cancer.

In some embodiments, the disease or disorder is uveal melanoma.

In some embodiments, the disease or disorder is chemotherapy-induced muscle toxicity and/or chemotherapy-induced cardio-toxicity. In some embodiments, the disease or disorder is chemotherapy-induced neuropathy.

In some embodiments, the disease or disorder is fragile X syndrome.

In some embodiments, the disease or disorder is an autoimmune disorder. Non-limiting examples of an autoimmune disorder include rheumatoid arthritis, IgA nephropathy, vascular disease associated with kidney disease, systemic lupus erythematosus (SLE), Wegener's granulomatosis, relapsing polychondritis, atopic dermatitis, psoriasis, sarcoidosis, Behçet's disease, Vogt-Koyanagi-Harada's disease, uveitis, and idiopathic pulmonary fibrosis.

In some embodiments, the disease or disorder is a microorganism infection. In some embodiments, the microorganism infection is caused by virus, bacteria, fungus, or any combination of two or more thereof.

In some embodiments, the disease or disorder is sepsis and/or septic shock.

In some embodiments, the disease or disorder is severe viral-induced pneumonia. In some embodiments, the severe viral-induced pneumonia is associated with an infection by a respiratory virus. In some embodiments, the respiratory virus is selected from an influenza virus, a respiratory syncytial virus, a coronavirus, a rhinovirus, an adenovirus, and a parainfluenza virus. In some embodiments, the coronavirus is COVID-19.

In some embodiments, the disease or disorder is mild to severe acute respiratory distress syndrome (ARDS) wherein the subject has positive end-expiratory pressure (PEEP)≥5 cm H₂O; and PaO₂/FiO₂<300 mm Hg. In some embodiments, the subject has positive end-expiratory pressure (PEEP)≥5 cm H₂O; and PaO₂/FiO₂<200 mm Hg. In some embodiments, the ARDS is associated with an infection by a respiratory virus. In some embodiments, the respiratory virus is selected from an influenza virus, a respiratory syncytial virus, a coronavirus, a rhinovirus, an adenovirus, and a parainfluenza virus. In some embodiments, the coronavirus is COVID-19.

In some embodiments, the disease or disorder is acute lung injury. In some embodiments, the lung injury is induced by aspiration, trauma, pancreatitis, blood transfusion, or smoke or toxic chemical inhalation. In some embodiments, the lung injury is induced by a chemical selected from chlorine, sulfur mustard gas, phosgene, Lewisite, hydrogen chloride, sulfur dioxide, hydrogen sulfide, nitrogen dioxide, ammonia, hydrofluoric acid, ozone, methyl isocyanate, and a combination of two or more thereof. In some embodiments, the lung injury comprises chemical burns, pulmonary edema, laryngeal edema, lung tissue apoptosis, pneumonia, pneumonitis, bronchitis, bronchiolitis, fibrosis, acute respiratory distress syndrome, respiratory tract spasm, or a combination of two or more thereof.

In some embodiments, the disease or disorder is chemical-induced lung injury. In some embodiments, the lung injury is induced by a chemical selected from chlorine, sulfur mustard gas, phosgene, Lewisite, hydrogen chloride, sulfur dioxide, hydrogen sulfide, nitrogen dioxide, ammonia, hydrofluoric acid, ozone, methyl isocyanate, and a combination of two or more thereof. In some embodiments, the lung injury comprises chemical burns, pulmonary edema, laryngeal edema, lung tissue apoptosis, pneumonia, pneumonitis, bronchitis, bronchiolitis, fibrosis, acute respiratory distress syndrome, respiratory tract spasm, or a combination of two or more thereof.

In another aspect, any one of the compositions described herein are used in a method of preventing metastasis of a cancer in a subject in need thereof, the method comprising administering to the subject a therapeutically effective amount of the composition described herein.

In another aspect, any one of the compositions described herein are used in a method of ameliorating metastasis of a cancer in a subject in need thereof, the method comprising administering to the subject a therapeutically effective amount of the composition described herein.

In another aspect, any one of the compositions described herein are used in a method of minimizing metastasis of a cancer in a subject in need thereof, the method comprising administering to the subject a therapeutically effective amount of the composition described herein.

In another aspect, any one of the compositions described herein are used in a method of preventing relapse of a cancer in a subject in need thereof, the method comprising administering to the subject a therapeutically effective amount of the composition described herein.

In another aspect, any one of the compositions described herein are used in a method of ameliorating relapse of a cancer in a subject in need thereof, the method comprising administering to the subject a therapeutically effective amount of the composition described herein.

In another aspect, any one of the compositions described herein are used in a method of minimizing risk of relapse or delaying relapse of a cancer in a subject in need thereof, the method comprising administering to the subject a therapeutically effective amount of the composition described herein.

In another aspect, any one of the compositions described herein are used in a method of suppressing myeloid-derived suppressor cells (MDSCs) in a patient diagnosed with cancer or suffering therefrom, the method comprising administering to the patient a therapeutically effective amount of the composition described herein.

In another aspect, any one of the compositions described herein are used in a method of reducing immune suppression in a patient diagnosed with cancer or suffering therefrom, the method comprising administering to the patient a therapeutically effective amount of the composition described herein.

In another aspect, any one of the compositions described herein are used in a method of reducing regulatory T-cell count in a patient diagnosed with cancer or suffering therefrom, the method comprising administering to the patient a therapeutically effective amount of the composition described herein.

In another aspect, any one of the compositions described herein are used in a method of increasing CD4+ T-cell count in a patient diagnosed with cancer or suffering therefrom, the method comprising administering to the patient a therapeutically effective amount of the composition described herein.

In some embodiments, the cancer is a cancer of the circulatory system selected from angiosarcoma, fibrosarcoma, rhabdomyosarcoma, liposarcoma, myxoma, rhabdomyoma, fibroma, lipoma and teratoma, cancer of the mediastinum and pleura, and a vascular tumor; a cancer of the respiratory tract selected from cancer of the nasal cavity and middle ear, cancer of accessory sinuses, cancer of the larynx, cancer of the trachea, cancer of the bronchus and lung, small cell lung cancer (SCLC), non-small cell lung cancer (NSCLC), bronchogenic carcinoma, squamous cell carcinoma, undifferentiated small cell carcinoma, undifferentiated large cell carcinoma, adenocarcinoma, alveolar (bronchiolar) carcinoma, bronchial adenoma, sarcoma, lymphoma, chondromatous hamartoma, and mesothelioma; a cancer of the gastrointestinal system selected from squamous cell carcinoma, adenocarcinoma, leiomyosarcoma, lymphoma, carcinoma, leiomyosarcoma, ductal adenocarcinoma, insulinoma, glucagonoma, gastrinoma, carcinoid tumors, vipoma, adenocarcinoma, carcinoid tumors, Karposi's sarcoma, leiomyoma, hemangioma, lipoma, neurofibroma, fibroma, adenocarcinoma, tubular adenoma, villous adenoma, hamartoma, and leiomyoma; a cancer of the genitourinary tract selected from adenocarcinoma, Wilm's tumor (nephroblastoma), lymphoma, leukemia, squamous cell carcinoma, transitional cell carcinoma, adenocarcinoma, adenocarcinoma, sarcoma of the prostate, seminoma, teratoma, embryonal carcinoma, teratocarcinoma, choriocarcinoma, interstitial cell carcinoma, fibroma, fibroadenoma, adenomatoid tumors, and lipoma; a cancer of the hepatobiliary and pancreatic system selected from hepatoma (hepatocellular carcinoma), cholangiocarcinoma, hepatoblastoma, angiosarcoma, hepatocellular adenoma, hemangioma, pheochromocytoma, insulinoma, vasoactive intestinal peptide tumor, islet cell tumor, pancreatic exocrine tumors (e.g., adenocarcinoma, acinar cell carcinoma, intraductal papillary-mucinous neoplasm, mucinous cystic neoplasm with an invasive adenocarcinoma), and pancreatic neuroendocrine tumors (e.g., gastrinoma, glucaganoma, insulinoma, somatostatinoma, VIPoma (vasoactive intestinal peptide), nonfunctional islet cell tumor); a cancer of the bone selected from osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors; a cancer of the nervous system selected from primary CNS lymphoma, osteoma, hemangioma, granuloma, xanthoma, osteitis deformans, meningioma, meningiosarcoma, gliomatosis, astrocytoma, medulloblastoma, glioma, ependymoma, germinoma (pinealoma), oligodendroglioma, schwannoma, retinoblastoma, congenital tumors, spinal cord neurofibroma, meningioma, glioma, and sarcoma; a cancer of the reproductive system selected from endometrial carcinoma, cervical carcinoma, pre-tumor cervical dysplasia, ovarian carcinoma, serous cystadenocarcinoma, mucinous cystadenocarcinoma, unclassified carcinoma, granulosa-thecal cell tumors, Sertoli-Leydig cell tumors, dysgerminoma, malignant teratoma, squamous cell carcinoma of the vulva, intraepithelial carcinoma of the vulva, adenocarcinoma of the vulva, fibrosarcoma of the vulva, melanoma of the vulva, vaginal clear cell carcinoma, vaginal squamous cell carcinoma, vaginal botryoid sarcoma (embryonal rhabdomyosarcoma), carcinoma of the fallopian tubes placental cancer, uterine cancer, penile cancer, prostate cancer, and testicular cancer; a cancer of the hematologic system selected from myeloid, acute lymphoblastic leukemia, chronic lymphocytic leukemia, myeloproliferative diseases, multiple myeloma, myelodysplastic syndrome, Hodgkin's disease, and non-Hodgkin's lymphoma; a cancer of the oral cavity selected from lip cancer, tongue cancer, gum cancer, floor of mouth cancer, palate cancer, parotid gland cancer, salivary gland cancer, tonsil cancer, cancer of the oropharynx, cancer of the nasopharynx, pyriform sinus cancer, and cancer of the hypopharynx; a cancer of the skin selected from malignant melanoma, cutaneous melanoma, basal cell carcinoma, squamous cell carcinoma, Karposi's sarcoma, moles dysplastic nevi, lipoma, angioma, dermatofibroma, and keloidal cancer; or a cancer selected from cancer of the adrenal glands, neuroblastoma, cancer of connective and soft tissue, cancer of the retroperitoneum and peritoneum, eye cancer, intraocular melanoma, uveal melanoma, cancer of adnexa, breast cancer, head or/and neck cancer, anal cancer, thyroid cancer, parathyroid cancer, cancer of the adrenal gland, cancer of the endocrine glands and related structures, secondary and unspecified malignant neoplasm of lymph nodes, secondary malignant neoplasm of respiratory and digestive systems, and secondary malignant neoplasm of other sites. In some embodiments, the cancer is glioblastoma multiforme (GBM). In some embodiments, the cancer is not glioblastoma multiforme (GBM).

In another aspect, any one of the compositions described herein are used in a method of suppressing myeloid-derived suppressor cells (MDSCs) in a patient diagnosed with microorganism infection or suffering therefrom, the method comprising administering to the patient a therapeutically effective amount of the composition described herein.

In another aspect, any one of the compositions described herein are used in a method of reducing immune suppression in a patient diagnosed with microorganism infection or suffering therefrom, the method comprising administering to the patient a therapeutically effective amount of the composition described herein.

In another aspect, any one of the compositions described herein are used in a method of reducing regulatory T-cell count in a patient diagnosed with microorganism infection or suffering therefrom, the method comprising administering to the patient a therapeutically effective amount of the composition described herein.

In another aspect, any one of the compositions described herein are used in a method of increasing CD4+ T-cell count in a patient diagnosed with microorganism infection or suffering therefrom, the method comprising administering to the patient a therapeutically effective amount of the composition described herein.

In another aspect, any one of the compositions described herein are used in a method of suppressing myeloid-derived suppressor cells (MDSCs) in a patient diagnosed with sepsis or suffering therefrom, the method comprising administering to the patient a therapeutically effective amount of the composition described herein.

In another aspect, any one of the compositions described herein are used in a method of reducing immune suppression in a patient diagnosed with sepsis or suffering therefrom, the method comprising administering to the patient a therapeutically effective amount of the composition described herein.

In another aspect, any one of the compositions described herein are used in a method of reducing regulatory T-cell count in a patient diagnosed with sepsis or suffering therefrom, the method comprising administering to the patient a therapeutically effective amount of the composition described herein.

In another aspect, any one of the compositions described herein are used in a method of increasing CD4+ T-cell count in a patient diagnosed with sepsis or suffering therefrom, the method comprising administering to the patient a therapeutically effective amount of the composition described herein.

### Methods of Administration

In another aspect, the present disclosure is directed to oral administration of ibudilast, or pharmaceutically acceptable salt thereof. In terms of patient compliance and ease of administration, such an approach may be preferred, since patients are often averse to taking multiple pills or dosage forms, often multiple times daily, over the duration of treatment. In some embodiments, the ibudilast is administered in a single daily dosage form. In some embodiments, the single daily dosage form is a capsule.

### Dosages

Therapeutic amounts can be empirically determined and will vary with the particular condition being treated, the subject, and the efficacy and toxicity of each of the active agents contained in the composition. The actual dose to be administered will vary depending upon the age, weight, and general condition of the subject as well as the severity of the condition being treated, the judgment of the health care professional, and particular combination being administered.

Therapeutically effective amounts can be determined by those skilled in the art, and will be adjusted to the requirements of each particular case. Generally, a therapeutically effective amount of ibudilast or pharmaceutically acceptable salt thereof will range from a total daily dosage of about.

In some embodiments, the therapeutically effective amount of ibudilast or pharmaceutically acceptable salt thereof is at least about 5 mg, at least about 10 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg. In some embodiments, the therapeutically effective amount of ibudilast or pharmaceutically acceptable salt thereof is at least about 50 mg.

In some embodiments, the therapeutically effective amount of ibudilast or pharmaceutically acceptable salt thereof is from about 5 mg to about 10 mg, 5 mg to about 20 mg, 5 mg to about 30 mg, 5 mg to about 40 mg, 5 mg to about 50 mg, 5 mg to about 60 mg, 5 mg to about 70 mg, 5 mg to about 80 mg, 5 mg to about 90 mg, 5 mg to about 100 mg, 5 mg to about 150 mg, 5 mg to about 200 mg, 10 mg to about 20 mg, 10 mg to about 30 mg, 10 mg to about 40 mg, 10 mg to about 50 mg, 10 mg to about 60 mg, 10 mg to about 70 mg, 10 mg to about 80 mg, 10 mg to about 90 mg, 10 mg to about 100 mg, 10 mg to about 150 mg, 10 mg to about 200 mg, 20 mg to about 30 mg, 20 mg to about 40 mg, 20 mg to about 50 mg, 20 mg to about 60 mg, 20 mg to about 70 mg, 20 mg to about 80 mg, 20 mg to about 90 mg, 20 mg to about 100 mg, 20 mg to about 250 mg, 20 mg to about 200 mg, 30 mg to about 40 mg, 30 mg to about 50 mg, 30 mg to about 60 mg, 30 mg to about 70 mg, 30 mg to about 80 mg, 30 mg to about 90 mg, 30 mg to about 100 mg, 30 mg to about 250 mg, 30 mg to about 200 mg, 40 mg to about 50 mg, 40 mg to about 60 mg, 40 mg to about 70 mg, 40 mg to about 80 mg, 40 mg to about 90 mg, 40 mg to about 100 mg, 40 mg to about 250 mg, 40 mg to about 200 mg, 50 mg to about 60 mg, 50 mg to about 70 mg, 50 mg to about 80 mg, 50 mg to about 90 mg, 50 mg to about 100 mg, 50 mg to about 250 mg, or 50 mg to about 200 mg. In some embodiments, the therapeutically effective amount of ibudilast or pharmaceutically acceptable salt thereof is from about 10 mg to about 100 mg.

In some embodiments, the therapeutically effective amount of ibudilast or pharmaceutically acceptable salt thereof is about 5 mg, about 10 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, or about 200 mg. In some embodiments, the therapeutically effective amount of ibudilast or pharmaceutically acceptable salt thereof is about 50 mg.

In some embodiments, the therapeutically effective amount of ibudilast or pharmaceutically acceptable salt thereof is at least about 5 mg/day, at least about 10 mg/day, at least about 20 mg/day, at least about 25 mg/day, at least about 30 mg/day, at least about 35 mg/day, at least about 40 mg/day, at least about 45 mg/day, at least about 50 mg/day, at least about 55 mg/day, at least about 60 mg/day, at least about 65 mg/day, at least about 70 mg/day, at least about 75 mg/day, at least about 80 mg/day, at least about 85 mg/day, at least about 90 mg/day, at least about 95 mg/day, at least about 100 mg/day, at least about 110 mg/day, at least about 120 mg/day, at least about 130 mg/day, at least about 140 mg/day, at least about 150 mg/day, at least about 160 mg/day, at least about 170 mg/day, at least about 180 mg/day, at least about 190 mg/day, or at least about 200 mg/day. In some embodiments, the therapeutically effective amount of ibudilast or pharmaceutically acceptable salt thereof is at least about 50 mg/day.

In some embodiments, the therapeutically effective amount of ibudilast or pharmaceutically acceptable salt thereof is from about 5 mg/day to about 10 mg/day, 5 mg/day to about 20 mg/day, 5 mg/day to about 30 mg/day, 5 mg/day to about 40 mg/day, 5 mg/day to about 50 mg/day, 5 mg/day to about 60 mg/day, 5 mg/day to about 70 mg/day, 5 mg/day to about 80 mg/day, 5 mg/day to about 90 mg/day, 5 mg/day to about 100 mg/day, 5 mg/day to about 150 mg/day, 5 mg/day to about 200 mg/day, 10 mg/day to about 20 mg/day, 10 mg/day to about 30 mg/day, 10 mg/day to about 40 mg/day, 10 mg/day to about 50 mg/day, 10 mg/day to about 60 mg/day, 10 mg/day to about 70 mg/day, 10 mg/day to about 80 mg/day, 10 mg/day to about 90 mg/day, 10 mg/day to about 100 mg/day, 10 mg/day to about 150 mg/day, 10 mg/day to about 200 mg/day, 20 mg/day to about 30 mg/day, 20 mg/day to about 40 mg/day, 20 mg/day to about 50 mg/day, 20 mg/day to about 60 mg/day, 20 mg/day to about 70 mg/day, 20 mg/day to about 80 mg/day, 20 mg/day to about 90 mg/day, 20 mg/day to about 100 mg/day, 20 mg/day to about 250 mg/day, 20 mg/day to about 200 mg/day, 30 mg/day to about 40 mg/day, 30 mg/day to about 50 mg/day, 30 mg/day to about 60 mg/day, 30 mg/day to about 70 mg/day, 30 mg/day to about 80 mg/day, 30 mg/day to about 90 mg/day, 30 mg/day to about 100 mg/day, 30 mg/day to about 250 mg/day, 30 mg/day to about 200 mg/day, 40 mg/day to about 50 mg/day, 40 mg/day to about 60 mg/day, 40 mg/day to about 70 mg/day, 40 mg/day to about 80 mg/day, 40 mg/day to about 90 mg/day, 40 mg/day to about 100 mg/day, 40 mg/day to about 250 mg/day, 40 mg/day to about 200 mg/day, 50 mg/day to about 60 mg/day, 50 mg/day to about 70 mg/day, 50 mg/day to about 80 mg/day, 50 mg/day to about 90 mg/day, 50 mg/day to about 100 mg/day, 50 mg/day to about 250 mg/day, or 50 mg/day to about 200 mg/day. In some embodiments, the therapeutically effective amount of ibudilast or pharmaceutically acceptable salt thereof is from about 10 mg/day to about 100 mg/day.

In some embodiments, the therapeutically effective amount of ibudilast or pharmaceutically acceptable salt thereof is about 5 mg/day, about 10 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, or about 200 mg/day. In some embodiments, the therapeutically effective amount of ibudilast or pharmaceutically acceptable salt thereof is about 50 mg/day.

Depending upon the dosage amount and precise condition to be treated, administration can be one, two, three, or four times daily for a time course of one day to several days, weeks, months, and even years, and may even be for the life of the patient. Illustrative dosing regimens will last a period of at least about a week, from about 1-4 weeks, from about 1-8 weeks, from 1-12 weeks, from 1-16 weeks, from 1-20 weeks, from 1-24 weeks, from 1-36 weeks, from 1-48 weeks, from 1-52 weeks, from 1-60 weeks, from 1-72 weeks, from 1-84 weeks, from 1-96 weeks, from 1 week to 1 year, from 1 week to 2 years, from 1 week to 3 years, from 1 week to 4 years, from 1 week to 5 years, or longer. In some embodiments, the dosing regimen is for a period of at least about 12, 24, 36, 48, 60, 72, 84, or 96 weeks. In some embodiments, the dosing regimen is for a period of about 12, 24, 36, 48, 60, 72, 84, or 96 weeks. In some embodiments, the dosing regimen is for a period of at least about 1 year, 2 years, 3 years, 4 years, or 5 years. In some embodiments, the dosing regimen is for a period of about 1 year, 2 years, 3 years, 4 years, or 5 years.

Practically speaking, a unit dose of any given composition of the disclosure or active agent can be administered in a variety of dosing schedules, depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth.

### Other Actives

A formulation (or kit) in accordance with the disclosure may contain, in addition to ibudilast or a pharmaceutically acceptable salt thereof, one or more additional active agents. In some embodiments, the one or more other therapeutic agent is one that possesses a mechanism of action different from that of ibudilast. Such active ingredients can be found listed in the FDA's Orange Book, Goodman & Gilman The Pharmacological Basis of Therapeutics, J. Griffith Hardman, L. L. Limbird, A. Gilman, 11th Ed., 2005, The Merck Manual, 18th edition, 2007, and The Merck Manual of Medical Information 2003. In some embodiments, ibudilast is the sole active agent in the pharmaceutical composition.

### Kits

Also provided herein is a kit containing any one of the compositions of the disclosure, accompanied by instructions for use.

For example, the kit comprises ibudilast, or pharmaceutically acceptable salt thereof, along with instructions for use. The ibudilast, or pharmaceutically acceptable salt thereof, and may be packaged in any manner suitable for administration, so long as the packaging, when considered along with the instructions for administration, clearly indicates the manner in which the drug components is to be administered.

For example, in an illustrative kit comprising ibudilast, or pharmaceutically acceptable salt thereof, the kit may be organized by any appropriate time period, such as by day. As an example, for Day 1, a representative kit may comprise unit dosages of each of ibudilast, or pharmaceutically acceptable salt thereof. If each of the drugs is to be administered twice daily, then the kit may contain, corresponding to Day 1, two rows of unit dosage forms of each of ibudilast, or pharmaceutically acceptable salt thereof, along with instructions for the timing of administration. Alternatively, if ibudilast, or pharmaceutically acceptable salt thereof, differ in the timing or quantity of administration, then such would be reflected in the packaging and instructions. Various embodiments according to the above may be readily envisioned, and would of course depend upon the particular combination of drugs, in addition to ibudilast, or pharmaceutically acceptable salt thereof, employed for treatment, their corresponding dosage forms, recommended dosages, intended patient population, and the like. The packaging may be in any form commonly employed for the packaging of pharmaceuticals, and may utilize any of a number of features such as different colors, wrapping, tamper-resistant packaging, blister packs, dessicants, and the like.

It is to be understood that while the disclosure has been described in conjunction with preferred specific embodiments, the foregoing description as well as the examples that follow are intended to illustrate and not limit the scope of the disclosure. Other aspects, advantages and modifications within the scope of the disclosure will be apparent to those skilled in the art to which the disclosure pertains.

### EXAMPLES

### Example 1. Drug Pellet Formation (Batch 1)

| Ingredient | Wt. % drug pellet |
|---|---|
| sugar sphere | 65.11 |
| ibudilast | 21.87 |
| hypromellose (METHOCEL ™ E5 Premium LV) | 7.38 |
| silicon dioxide (SYLOID ® 244 FP) | 5.64 |

Drug loading onto the sugar sphere was accomplished by wurster coating. A mixture of hypromellose, ibudilast, and silicon dioxide in water was sprayed onto the sugar sphere. Water used for the coating was subsequently removed from the drug pellet product.

### Example 2. Drug Pellet Formation (Batch 2)

| Ingredient | Wt. % drug pellet |
|---|---|
| sugar sphere | 65.22 |
| ibudilast | 21.74 |
| hypromellose (METHOCEL ™ E5 Premium LV) | 7.39 |
| silicon dioxide (SYLOID ® 244 FP) | 5.65 |

Drug loading onto the sugar sphere was accomplished by wurster coating. A mixture of hypromellose, ibudilast, and silicon dioxide in purified water was sprayed onto the sugar sphere. Water used for the coating was subsequently removed from the drug pellet product.

### Example 3. Extended-Release (ER) Coating (2:1, Wt. % Ethyl Cellulose to Wt. % HPMC) of Drug Pellet

| Ingredient | Wt. % ER pellet |
|---|---|
| Drug pellet (from Example 1) | 90.91 |
| Ethyl cellulose | 4.50 |
| Hypromellose (METHOCEL ™ E5 Premium LV) | 2.25 |
| Triethyl citrate | 0.32 |
| Talc | 2.02 |

Coating of the drug pellet was accomplished by wurster coating. A mixture of ethyl cellulose, hypromellose, triethyl citrate, and talc in isopropyl alcohol and methylene chloride was sprayed onto the drug pellet. Solvents (isopropyl alcohol and methylene chloride) used for the coating were subsequently removed from the coated pellet product (ER coated pellets).

### Example 4. Extended-Release (ER) Coating (2:1, Wt. % Ethyl Cellulose to Wt. % HPMC) of Drug Pellet

| Ingredient | Wt. % ER pellet |
|---|---|
| Drug pellet (from Example 2) | 94.57 |
| Ethyl cellulose (ETHOCEL ™ Standard 7 Premium) | 2.69 |
| Hypromellose (METHOCEL ™ E5 Premium LV) | 1.34 |
| Triethyl citrate | 0.19 |
| Talc | 1.21 |

Coating of the drug pellet was accomplished by wurster coating. A mixture of ethyl cellulose, hypromellose, triethyl citrate, and talc in isopropyl alcohol and methylene chloride was sprayed onto the drug pellet. Solvents (isopropyl alcohol and methylene chloride) used for the coating were subsequently removed from the coated pellet product (ER coated pellets).

### Example 5. Extended-Release (ER) Coating (1:1, Wt. % Ethyl Cellulose to Wt. % HPMC) of Drug Pellet

| Ingredient | Wt. % ER pellet |
|---|---|
| Drug pellet (from Example 1) | 90.91 |
| Ethyl cellulose | 3.40 |
| Hypromellose (METHOCEL ™ E5 Premium LV) | 3.40 |
| Triethyl citrate | 0.24 |
| Talc | 2.04 |

Coating of the drug pellet was accomplished by wurster coating. A mixture of ethyl cellulose, hypromellose, triethyl citrate, and talc in isopropyl alcohol and methylene chloride was sprayed onto the drug pellet. Solvents (isopropyl alcohol and methylene chloride) used for the coating were subsequently removed from the coated pellet product (ER coated pellets).

### Example 6. Extended-Release (ER) Coating (1:1, Wt. % Ethyl Cellulose to Wt. % HPMC) of Drug Pellet

| Ingredient | Wt. % ER pellet |
|---|---|
| Drug pellet (from Example 2) | 94.34 |
| Ethyl cellulose | 2.12 |
| Hypromellose (METHOCEL ™ E5 Premium LV) | 2.12 |
| Triethyl citrate | 0.15 |
| Talc | 1.27 |

Coating of the drug pellet was accomplished by wurster coating. A mixture of ethyl cellulose, hypromellose, triethyl citrate, and talc in isopropyl alcohol and methylene chloride was sprayed onto the drug pellet. Solvents (isopropyl alcohol and methylene chloride) used for the coating were subsequently removed from the coated pellet product (ER coated pellets).

### Example 7. Extended-Release (ER) Coating (1:2, Wt. % Ethyl Cellulose to Wt. % HPMC) of Drug Pellet

| Ingredient | Wt. % ER pellet |
|---|---|
| Drug pellet (from Example 1) | 93.02 |
| Ethyl cellulose | 1.76 |
| Hypromellose (METHOCEL ™ E5 Premium LV) | 3.51 |
| Triethyl citrate | 0.12 |
| Talc | 1.58 |

Coating of the drug pellet was accomplished by wurster coating. A mixture of ethyl cellulose, hypromellose, triethyl citrate, and talc in isopropyl alcohol and methylene chloride was sprayed onto the drug pellet. Solvents (isopropyl alcohol and methylene chloride) used for the coating were subsequently removed from the coated pellet product (ER coated pellets).

### Example 8. Extended-Release (ER) Coating (1:2, Wt. % Ethyl Cellulose to Wt. % HPMC) of Drug Pellet

| Ingredient | Wt. % ER pellet |
|---|---|
| Drug pellet (from Example 2) | 93.98 |
| Ethyl cellulose (ETHOCEL ™ Standard 7 Premium) | 1.52 |
| Hypromellose (METHOCEL ™ E5 Premium LV) | 3.03 |
| Triethyl citrate | 0.11 |
| Talc | 1.36 |

Coating of the drug pellet was accomplished by wurster coating. A mixture of ethyl cellulose, hypromellose, triethyl citrate, and talc in isopropyl alcohol and methylene chloride was sprayed onto the drug pellet. Solvents (isopropyl alcohol and methylene chloride) used for the coating were subsequently removed from the coated pellet product (ER coated pellets).

### Example 9. Extended-Release (ER) Coating (10:16, Wt. % Ethyl Cellulose to Wt. % HPMC) of Drug Pellet

| Ingredient | Wt. % ER pellet |
|---|---|
| Drug pellet (from Example 2) | 88.50 |
| Ethyl cellulose (ETHOCEL ™ Standard 7 Premium) | 3.33 |
| Hypromellose (METHOCEL ™ E5 Premium LV) | 5.34 |
| Triethyl citrate | 0.23 |
| Talc | 2.60 |

Coating of the drug pellet was accomplished by wurster coating. A mixture of ethyl cellulose, hypromellose, triethyl citrate, and talc in isopropyl alcohol and methylene chloride was sprayed onto the drug pellet. Solvents (isopropyl alcohol and methylene chloride) used for the coating were subsequently removed from the coated pellet product (ER coated pellets).

### Example 10. Delayed Release (DR) Coating of Drug Pellet

| Ingredient | Wt. % DR pellet |
|---|---|
| Drug pellet (from Example 1) | 79.96 |
| EUDRAGIT ® L30 D55 | 12.52 |
| Triethyl citrate | 1.25 |
| Talc | 6.26 |

Coating of the drug pellet was accomplished by wurster coating. A mixture of EUDRAGIT® L30 D55, triethyl citrate, and talc in purified water was sprayed onto the drug pellet. Water used for the coating was subsequently removed from the coated pellet product (DR coated pellets).

### Example 11. Delayed Release (DR) Coating of Drug Pellet

| Ingredient | Wt. % DR pellet |
|---|---|
| Drug pellet (from Example 2) | 87.41 |
| EUDRAGIT ® L30 D55 | 7.87 |
| Triethyl citrate | 0.79 |
| Talc | 3.93 |

Coating of the drug pellet was accomplished by wurster coating. A mixture of EUDRAGIT® L30 D55, triethyl citrate, and talc in purified water was sprayed onto the drug pellet. Water used for the coating was subsequently removed from the coated pellet product (DR coated pellets).

### Example 12. Dissolution Study of ER and DR Coated Pellets

The DR coated pellets from Example 10 and ER coated pellets from Examples 3, 5, 7, and 9 were compared to PINATOS® (a previously commercially available intermediate release formulation of ibudilast) under dissolution conditions (USP-II, 900 mL, 100 rpm), wherein the dissolution media was: pH 6.8 phosphate buffer (25 mM). Results are shown in the table below.

| | Percent dissolution | | | | | | | | |
|---|---|---|---|---|---|---|---|---|---|
| time | | DR | ER pellets | | | | | | 1:1 ER/DR** |
| point (h) | control* | pellets | A | B | C | D1 | D2 | D3 | pellets |
| 0 | 0 | 0 | 0 | 0 | 0 | nd | nd | nd | 0 |
| 2 | 43 | 64 | 15 | 12 | 22 | 21 | 24 | 20 | 42 |
| 4 | 60 | 95 | 25 | 21 | 40 | 38 | 41 | 37 | 61 |
| 6 | 70 | 99 | 34 | 29 | 54 | 54 | 56 | 53 | 68 |
| 8 | 76 | 99 | 42 | 36 | 65 | 67 | 72 | 66 | 73 |
| 10 | 79 | 100 | 49 | 43 | 75 | 78 | 84 | 77 | 77 |
| 12 | 82 | 100 | 55 | 49 | 83 | 83 | 91 | 82 | 81 |
| 18 | 84 | 100 | 70 | 65 | 94 | nd | 97 | nd | 88 |

*control = PINATOS ® formulation;
**ER pellets from Example 5;
nd = not determined
A = ER pellets (example 3);
B = ER pellets (example 5);
C = ER pellets (example 7);
D1 = ER pellets (example 9);
D2 and D3 = additional batches of ER pellets from example 9

### Example 13. Dissolution Study of Select Combinations of ER and DR Coated Pellets

The DR coated pellets from Example 10 and ER coated pellets from Examples 3, 7, and 9 were compared to PINATOS® (a previously commercially available intermediate release formulation of ibudilast) under the dissolution conditions described in Example 11. Results are shown in the table below.

| | Percent dissolution | | | | | | |
|---|---|---|---|---|---|---|---|
| time | | combo | combo | combo | combo | combo | combo |
| point (h) | control* | 1 | 2 | 3 | 4 | 5 | 6 |
| 2 | 43 | 31 | 39 | 34 | 29 | 49 | 50 |
| 4 | 60 | 51 | 53 | 48 | 45 | 59 | 57 |
| 6 | 70 | 66 | 63 | 59 | 58 | 63 | 62 |
| 8 | 76 | 78 | 72 | 71 | 69 | 67 | 66 |
| 10 | 79 | 88 | 79 | 79 | 78 | 71 | 70 |
| 12 | 82 | 93 | 85 | 86 | 86 | 74 | 73 |
| 18 | 84 | 96 | 94 | 94 | 96 | 82 | 82 |
| 24 | 85 | nd | nd | nd | nd | nd | nd |
| recovery | 86 | 97 | nd | nd | 98 | 88 | 84 |

*control = PINATOS ® formulation;
nd = not determined;
combo 1 = 90:10 of (number of ER coated pellets from Example 7):(number of DR coated pellets of Example 10);
combo 2 = 70:30 of (number of ER coated pellets from Example 9):(number of DR coated pellets of Example 10);
combo 3 = 80:20 of (number of ER coated pellets from Example 9):(number of DR coated pellets of Example 10);
combo 4 = 90:10 of (number of ER coated pellets from Example 9):(number of DR coated pellets of Example 10);
combo 5 = 50:50 of (number of ER coated pellets from Example 3):(number of DR coated pellets of Example 10);
combo 6 = 50:50 of (number of ER coated pellets from Example 3):(number of DR coated pellets of Example 10)

### Example 14. Dissolution Study of CAP1 (100% ER Coated Pellets)

The ER coated pellets from Example 6 were stored for up to 2 months at 40±2° C. and 75±5% RH, and assessed for their dissolution profile after storage at t=0, 1 month, and 2 months under the dissolution conditions as described in USP <711>. Percent dissolution at the 2 h, 6 h, and 18 h time-points are shown in the table below.

| | Percent dissolution (assessed by HPLC) | | | | | |
|---|---|---|---|---|---|---|
| | storage t = 0 | | storage t = 1 mo | | storage t = 2 mo | |
| 2 h | Min | 19% | Min | 21% | Min | 24% |
| | Max | 20% | Max | 23% | Max | 29% |
| | Mean | 20% | Mean | 22% | Mean | 27% |
| | % RSD | 2.9 | % RSD | 5.0 | % RSD | 7.4 |
| 6 h | Min | 47% | Min | 47% | Min | 51% |
| | Max | 49% | Max | 50% | Max | 57% |
| | Mean | 48% | Mean | 49% | Mean | 54% |
| | % RSD | 2.1 | % RSD | 2.5 | % RSD | 4.3 |
| 18 h | Min | 93% | Min | 89% | Min | 92% |
| | Max | 94% | Max | 92% | Max | 95% |
| | Mean | 94% | Mean | 91% | Mean | 94% |
| | % RSD | 0.6 | % RSD | 1.2 | % RSD | 1.1 |

### Example 15. Dissolution Study of CAP2 (80:20 ER to DR Coated Pellets)

A combination with 80:20 ratio of number of ER coated pellets from Example 6 to number of DR coated pellets from Example 11 were stored for 1 month at 40±2° C. and 75±5% RH, and assessed for their dissolution profile after storage at t=0 and 1 month under the dissolution conditions as described in USP <711>. Percent dissolution at the 2 h, 6 h, and 18 h time-points are shown in the table below.

| Percent dissolution (assessed by HPLC) | |
|---|---|
| storage t = 0 | storage t = 1 mo |

| | | | | |
|---|---|---|---|---|
| 2 h | Min | 35% | Min | 34% |
| | Max | 40% | Max | 38% |
| | Mean | 38% | Mean | 36% |
| | % RSD | 5.0 | % RSD | 4.6 |
| 6 h | Min | 60% | Min | 56% |
| | Max | 63% | Max | 60% |
| | Mean | 61% | Mean | 58% |
| | % RSD | 2.2 | % RSD | 2.5 |
| 18 h | Min | 92% | Min | 89% |
| | Max | 96% | Max | 92% |
| | Mean | 95% | Mean | 90% |
| | % RSD | 1.6 | % RSD | 1.1 |

### Example 16. Dissolution Study of CAP3 (50:50 ER to DR Coated Pellets)

A combination with 50:50 ratio of number of ER coated pellets from Example 4 to number of DR coated pellets from Example 11 were stored for up to 6 months at 40±2° C. and 75±5% RH, and assessed for their dissolution profile after storage at t=0, 1 month, 2 months, 3 months, and 6 months under the dissolution conditions as described in USP <711>. Percent dissolution at the 2 h, 6 h, and 18 h time-points are shown in the table below.

| | Percent dissolution (assessed by HPLC) | | | | | | | | | |
|---|---|---|---|---|---|---|---|---|---|---|
| | storage t = 0 | | storage t = 1 mo | | storage t = 2 mo | | storage t = 3 mo | | storage t = 6 mo | |
| 2 h | Min | 45% | Min | 49% | Min | 47% | Min | 44% | Min | 51% |
| | Max | 53% | Max | 53% | Max | 54% | Max | 53% | Max | 59% |
| | Mean | 50% | Mean | 51% | Mean | 51% | Mean | 49% | Mean | 55% |
| | % RSD | 5.4 | % RSD | 3.4 | % RSD | 4.8 | % RSD | 5.9 | % RSD | 5.6 |
| 6 h | Min | 63% | Min | 67% | Min | 64% | Min | 61% | Min | 67% |
| | Max | 71% | Max | 70% | Max | 71% | Max | 69% | Max | 74% |
| | Mean | 68% | Mean | 69% | Mean | 68% | Mean | 65% | Mean | 70% |
| | % RSD | 4.2 | % RSD | 1.8 | % RSD | 3.5 | % RSD | 4.0 | % RSD | 4.0 |
| 18 h | Min | 85% | Min | 88% | Min | 87% | Min | 80% | Min | 89% |
| | Max | 91% | Max | 90% | Max | 90% | Max | 87% | Max | 92% |
| | Mean | 90% | Mean | 89% | Mean | 89% | Mean | 83% | Mean | 90% |
| | % RSD | 2.6 | % RSD | 0.9 | % RSD | 1.3 | % RSD | 2.7 | % RSD | 1.5 |

### Example 17. Phase 1 Study to Examine Oral Modified-Release Formulations of Ibudilast in Healthy Volunteers

This is a Phase 1, single-center, randomized, open-label, parallel-group, crossover study to evaluate the pharmacokinetics and bioavailability of three different modified-release formulations (MRF):
CAP1: a composition as described in Example 14
CAP2: a composition as described in Example 15
CAP3: a composition as described in Example 16
   The study consists of two parts. In Part 1, study duration consists of up to a 28-day Screening Phase and a one Week Treatment Phase. In Part 2, study duration consists of up to a 28-day Screening Phase and an approximately 21-day Treatment Phase.

### Part 1 (Fasted State)

Thirty-six (n=12/group) healthy fasted subjects are randomly assigned to one of three modified-release capsule formulations (CAP1, CAP2, CAP3) administered as a single oral dose of 50 mg in the fasted state. Pre- and post-dose PK and safety assessments are conducted at regular intervals.

Subjects are to check in to the clinic on the evening of Day −1. Subjects are instructed to begin fasting for at least 10 hours prior to dosing on Day 1. Except for water given with the study drug, no fluids is allowed for 1 hour before dosing until 1 hour post-dose. Water is permitted at all other times. On the morning of dosing day (Day 1) fasting is continued until 4 hours post study drug dose. Subjects remain in the clinic until Day 2 and are discharged after the 32-hour PK sample has been collected. Subjects return to the clinic each morning on Days 3, 4, 5, 6, and 7 for a PK blood sample and vital sign assessment.

On Day 7 (End of Study visit), subjects are to have the final PK blood draw and scheduled assessments, and are discharged from the study. In total, blood samples (˜2 mL per sample) for PK sampling are collected within 30 minutes prior to dosing and at 0.5, 1, 2, 4, 6, 8, 10, 12, 24, 32, 48, 72, 96, 120, and 144 hours post-dose. PK analysis is performed and based on the results, one MRF (CAP1, CAP2, CAP3) is tested in Part 2.

### Part 2 (Fed and Fasted State)

An additional cohort of 12 subjects is randomly assigned to one of 2 sequences (n=6/sequence). A single dose of study drug is administered in a crossover fashion one week apart (washout period of 7 days between doses).

| Sequence | Week 1 | Week 2 | Week 3 |
|---|---|---|---|
| 1 (n = 6) | MRF fasted | MRF fed | IRC fasted |
| 2 (n = 6) | MRF fed | IRC fasted | MRF fasted |

IRC: immediate release composition (previously commercially available)

The MRF fasted group is to consist of a single dose of MRF 50 mg (1 capsule) administered in a fasted (without food) state. The MRF fed group is to consist of a single dose of MRF 50 mg (1 capsule) after consuming a high fat breakfast. The IRC fasted group is to consist of a single dose of 50 mg of the IRC (5×10 mg capsules) administered in a fasted state in both sequences (there is no IRC fed group in this study).

During each dosing sequence, subjects are to check-in to the study facility the night before dosing (Day −1) every week and remain at the facility until 32 hours after dosing. Subjects are discharged from the clinic on Day 2 after post-32 hours PK sample collection. Subjects are to return to the clinic to have the remaining PK sampling collection at 48 (Day 3), 72 (Day 4), and 96 (Day 5) hours after dosing. On Week 3 Day 8 (End of Study visit), subjects are to return to the facility to have the final PK blood draw (168 hours) and scheduled assessments. In total, blood samples (˜2 mL per sample) for PK sampling are collected within 45 minutes prior to dosing and at 0.5, 1, 2, 4, 6, 8, 10, 12, 24, 32, 48, 72, 96, and 168 hours post-dose (post 168 h for Week 3 only).

### Fasted Conditions (Part 1 and Part 2 Fasted)

Study drug is to be administered with a standardized volume of room temperature water (approximately 240 mL). Water is allowed as desired except for one hour before and one hour after drug administration and food is not allowed for at least 10 hours prior to dosing and at least 4 hours post-dose. Meals taken after dosing should be standardized in regard to composition and time of administration while housed in the clinic.

### Fed Conditions (Part 2 Fed)

After check-in at the clinic (Week 1 Day −1, Week 2 Day −1, Week 3 Day −1), subjects are instructed to begin fasting for at least 10 hours prior to dosing on Day 1.

Subjects dosed in the fed state should start the meal 30±2 minutes prior to administration of study drug and eat this meal within 30 minutes. The meal is to be a high-fat (approximately 50 percent of total caloric content of the meal) and high-calorie (approximately 800 to 1000 kcal) meal. This test meal should derive approximately 150, 250, and 500-600 kcal from protein, carbohydrate, and fat, respectively.

Except for water given with the study drug and fluids provided with the high-fat meal, no fluids are allowed for 1 hour before dosing until 1 hour post-dose.

Standard meals are to resume≥4 hours after the subject is given study drug in both fed and fasted states.

### Inclusion Criteria

In order to be eligible to participate in the study, a subject must meet all of the following criteria:
1. Able to provide written informed consent.
2. Healthy nonsmoking (no use of tobacco or nicotine products within 3 months prior to screening) male and female subjects ages 18 to 65 years old (inclusive)
3. Body mass index (BMI) of 18.5 kg/m² or greater, but less than 32 kg/m².
4. Normal renal function (eGFR [calculated using the CKD-EPI equation]≥60 L/min/1.73 m²)
5. Liver enzymes (ALT, AST, GGT) should be less than twice the upper limit of normal (ULN)
6. Screening electrocardiogram (ECG) with Fridericia's corrected QT interval (QTcF) within normal limits or judged by the Investigator as non-clinically significant
7. Agree to use barrier contraceptive methods during the study (hormonal contraceptive alone is not acceptable).
8. Females must have a negative urine pregnancy test at Screening and a negative serum pregnancy test on Study Day-1.
9. Females of non-childbearing potential must be:
a. post-menopausal (spontaneous amenorrhea for at least 12 months prior to first dosing) with confirmation by documented follicle-stimulating hormone (FSH) levels≥40 IU/L; or
b. surgically sterile (bilateral oophorectomy, bilateral salpingectomy, hysterectomy or tubal ligation) at least 3 months prior to first dosing.

### Exclusion Criteria

A subject who meets any of the following criteria will be excluded from participation in this study:
1. History of clinically significant drug allergy or anaphylaxis, including known hypersensitivity to ibudilast or its formulation components.
2. Positive cotinine test at screening and Day-1.
3. Any clinically significant abnormal finding at physical examination at Screening.
4. Clinically significant abnormal laboratory test results or positive serology test results for HBsAg, HCV antibody, or HIV antigen and antibody at Screening.
5. Clinically significant ECG abnormalities or vital signs abnormalities (systolic blood pressure lower than 90 or over 140 mmHg, diastolic blood pressure lower than 50 or over 90 mmHg, or heart rate less than 50 or over 100 bpm) at Screening.
6. Presence or history of significant gastrointestinal, liver or kidney disease, or surgery that may affect drug bioavailability.
7. Clinical evidence or a history of clinically significant cardiovascular, respiratory, renal, hepatic, gastrointestinal, hematological, neurologic, endocrine, metabolic, psychiatric disease or other chronic diseases as judged by the Investigator.
8. History of alcohol abuse within one year prior to Screening or regular use of alcohol within 6 months prior to Screening that exceeds 10 units for women or 15 units for men of alcohol per week (1 unit=340 mL of beer 5%, 140 mL of wine 12%, or 45 mL of distilled alcohol 40%); and/or history of drug abuse within one year prior to Screening or recreational use of soft drugs (such as marijuana) within 1 month or hard drugs (such as cocaine, phencyclidine [PCP], crack, opioid derivatives including heroin, and amphetamine derivatives) within 3 months prior to Screening.
9. Positive results on urine drug and alcohol screen at Screening visit and Day −1.
10. Platelets<100×10⁹/L at Screening or history of thrombocytopenia
11. Currently pregnant or nursing.
12. Donation of plasma within 7 days prior to first dosing or donation or loss of 500 mL or more of whole blood within 8 weeks prior to first dosing or have poor peripheral venous access.
13. Currently participating, or has participated in a study with an investigational or marketed compound or device within 30 days or 5 half-lives, whichever is longer, prior to first dosing or has participated in a study with a biological product within 90 days or 5 half-lives, whichever is longer, prior to first dosing.
14. Use of medications within the timeframes specified below:
   Depot injection or implant of any drug (with the exception of hormonal contraceptives) for 3 months prior to first dosing and throughout the study;
   Live attenuated vaccines for 1 month prior to first dosing and throughout the study, or any other vaccine, including COVID-19 vaccine, for 14 days prior to first dosing and throughout the study;
   Any drug known to induce or inhibit hepatic drug metabolism (with the exception of hormonal contraceptives), including St. John's wort, for 30 days prior to first dosing and throughout the study;
   Prescription medications for 14 days prior to first dosing and throughout the study;
   Over-the-counter (OTC) medications and natural health products (including herbal remedies, homeopathic and traditional medicines, probiotics, food supplements such as vitamins, minerals, amino acids, essential fatty acids, and protein supplements used in sports) for 7 days prior to first dosing (with the exception of the occasional use of acetaminophen up to 2 g daily) and throughout the study;
   Herbal laxative supplement (e.g., Cassia Senna) or any product that may affect drug absorption and distribution for 14 days prior to first dosing and throughout the study.
15. For Part 2 only: Subjects who cannot complete the high-fat meal within specified time and strict vegetarians/vegans.
16. Any reason which, in the opinion of the Investigator, would prevent the subject from participating in the study.
17. Any subject who has taken ibudilast previously.

### Example 18. Phase 1 Study to Examine Oral Modified-Release Formulations of Ibudilast in Healthy Volunteers

This was a Phase 1, single-center, randomized, open-label, parallel-group, crossover study to evaluate the pharmacokinetics and bioavailability of three different modified-release formulations (MRF):
CAP1: a composition as described in Example 14
CAP2: a composition as described in Example 15
CAP3: a composition as described in Example 16
   The study consisted of three parts. In Part 1, study duration consisted of up to a 28-day Screening Phase and a one Week Treatment Phase. In Part 2, study duration consisted of up to a 28-day Screening Phase and an approximately 21-day Treatment Phase. In Part 3, study duration consisted of up to 28-day Screening Phase and approximately 18 days Treatment Phase.

### Part 1 (Fasted State)

Thirty-six (n=12/group) healthy fasted subjects were randomly assigned to one of three modified-release capsule formulations (CAP1, CAP2, CAP3) administered as a single oral dose of 50 mg in the fasted state. Pre- and post-dose PK and safety assessments were conducted at regular intervals.

Subjects checked in to the clinic on the evening of Day −1. Subjects were instructed to begin fasting for at least 10 hours prior to dosing on Day 1. Except for water given with the study drug, no fluids was allowed for 1 hour before dosing until 1 hour post-dose. Water was permitted at all other times. On the morning of dosing day (Day 1) fasting was continued until 4 hours post study drug dose. Subjects remained in the clinic until Day 2 and were discharged after the 32-hour PK sample had been collected. Subjects returned to the clinic each morning on Days 3, 4, 5, 6, and 7 for a PK blood sample and vital sign assessment.

On Day 7 (End of Study visit), subjects had the final PK blood draw and scheduled assessments, and were discharged from the study. In total, blood samples (˜2 mL per sample) for PK sampling were collected within 30 minutes prior to dosing and at 0.5, 1, 2, 4, 6, 8, 10, 12, 24, 32, 48, 72, 96, 120, and 144 hours post-dose. PK analysis was performed and based on the results, one MRF (CAP1, CAP2, CAP3) was tested in Part 2.

PK results for CAP2 and CAP3 were similar, but CAP3 was brought forward due to its superior stability data (not shown) for further assessment in Part 2 of this study.

### Part 2 (Fed and Fasted State; Single Dose)

An additional cohort of 12 subjects was randomly assigned to one of 2 sequences (n=6/sequence). A single dose of study drug (MRF=CAP3) was administered in a crossover fashion one week apart (washout period of 7 days between doses).

| Sequence | Week 1 | Week 2 | Week 3 |
|---|---|---|---|
| 1 (n = 6) | MRF fasted | MRF fed | IRC fasted |
| 2 (n = 6) | MRF fed | IRC fasted | MRF fasted |

IRC: intermediate release composition (previously commercially available)

The MRF fasted group consisted of a single dose of MRF 50 mg (1 capsule) administered in a fasted (without food) state. The MRF fed group consisted of a single dose of MRF 50 mg (1 capsule) after consuming a high fat breakfast. The IRC fasted group consisted of a single dose of 50 mg of the IRC (5×10 mg capsules) administered in a fasted state in both sequences (there was no IRC fed group in this study).

During each dosing sequence, subjects checked in to the study facility the night before dosing (Day −1) every week and remained at the facility until 32 hours after dosing. Subjects were discharged from the clinic on Day 2 after post-32 hours PK sample collection. Subjects returned to the clinic to have the remaining PK sampling collection at 48 (Day 3), 72 (Day 4), and 96 (Day 5) hours after dosing. On Week 3 Day 8 (End of Study visit), subjects returned to the facility to have the final PK blood draw (168 hours) and scheduled assessments. In total, blood samples (˜2 mL per sample) for PK sampling were collected within 45 minutes prior to dosing and at 0.5, 1, 2, 4, 6, 8, 10, 12, 24, 32, 48, 72, 96, and 168 hours post-dose (post 168 h for Week 3 only).

### Part 3 (Fed and Fasted State; Multiple Dose)

An additional cohort of 16 (n=8/group) subjects was randomly assigned to one of 2 groups with different breakfast timing, BR-1 or BR-2. Study drug (MRF=CAP3) 50 mg capsule, was administered twice daily on Days 1 through 9 and a single dose was administered on Day 10. Subjects assigned to BR-1 received the study drug morning dose within 0.5 h of a standard breakfast. Subjects assigned to BR-2 received the study drug morning dose at least 1.5 h before a standard breakfast. All subjects received standardized meals throughout the rest of the day.

| Group | Breakfast/Morning dose Instruction |
|---|---|
| BR-1 (with | Morning dose administered within 0.5 h of a standard |
| breakfast) | breakfast. |
| BR-2 (without | Morning dose administered at least 1.5 h before a |
| breakfast) | standard breakfast. |

Subjects checked in to the clinic on the evening of Day-1. Subjects were instructed to begin fasting for at least 10 hours prior to dosing on Day 1. On Day 1, subjects were randomized to one of two groups, BR-1 (with breakfast) or BR-2 (without breakfast).

On Day 1, subjects started taking CAP350 mg (1 capsule) twice a day (b.i.d.) in AM and PM on Days 1 through 9 and a single dose on the morning of Day 10. Subjects remained in the clinic until Day 11. PK samples were collected on Day 1, Day 2, Day 5, Day 9, Day 10 and Day 11.

Subjects were discharged on Day 11 after the post-dose 32 hour PK sample collection. Subjects returned to the clinic to have the remaining PK sampling collection at post-dose 48 (Day 12), 72 (Day 13), and 96 (Day 14) hours after Day 10 AM dosing.

On Day 17 (End of Study visit), subjects returned to the clinic to have the last PK sample drawn (post-dose 168 hrs of Day 10 AM dose) and underwent End of Study assessments.

### Meal Schedule

### Fasted Conditions (Part 1 and Part 2 Fasted)

After check-in at the clinic, subjects were instructed to begin fasting for at least 10 hours prior to dosing on Day 1. Study drug was administered with a standardized volume of room temperature water. Food was not allowed at least 4 hours post-dose. Meals taken after dosing were standardized in regard to composition and time of administration while housed in the clinic.

### Fed Conditions (Part 2 Fed)

After check-in at the clinic, subjects were instructed to begin fasting for at least 10 hours prior to dosing on Day 1. Subjects dosed in the fed state started the meal 30±2 minutes prior to administration of study drug and ate this meal within 30 minutes. The meal was a high-fat (approximately 50 percent of total caloric content of the meal) and high-calorie (approximately 800 to 1000 kcal) meal. This test meal derived approximately 150, 250, and 500-600 kcal from protein, carbohydrate, and fat, respectively. Except for water given with the study drug and fluids provided with the high-fat meal, no fluids were allowed for 1 hour before dosing until 1 hour post-dose. Standard meals resumed≥4 hours after the subject was given study drug in both fed and fasted states.

Part 3 (“with Breakfast” and “without Breakfast”)

Subjects will be assigned to one of the 2 groups as follows:
BR-1 (with average breakfast, not high fat meal): Morning dose to be administered within 0.5 h of a standard breakfast.
BR-2 (without breakfast): Morning dose to be administered at least 1.5 h before a standard breakfast.
No meal restriction for evening dose, which was administered about 12 hours after the morning dose.

### Inclusion Criteria

In order to be eligible to participate in the study, a subject must have met all of the following criteria:
1. Able to provide written informed consent.
2. Healthy nonsmoking (no use of tobacco or nicotine products within 3 months prior to screening) male and female subjects ages 18 to 65 years old (inclusive)
3. Body mass index (BMI) of 18.5 kg/m² or greater, but less than 32 kg/m².
4. Normal renal function (eGFR [calculated using the CKD-EPI equation]≥60 L/min/1.73 m²)
5. Liver enzymes (ALT, AST, GGT) should be less than twice the upper limit of normal (ULN)
6. Screening electrocardiogram (ECG) with Fridericia's corrected QT interval (QTcF) within normal limits or judged by the Investigator as non-clinically significant
7. Agree to use barrier contraceptive methods during the study (hormonal contraceptive alone is not acceptable).
8. Females must have a negative urine pregnancy test at Screening and a negative serum pregnancy test on Study Day-1.
9. Females of non-childbearing potential must be:
   a. post-menopausal (spontaneous amenorrhea for at least 12 months prior to first dosing) with confirmation by documented follicle-stimulating hormone (FSH) levels≥40 IU/L; or
   b. surgically sterile (bilateral oophorectomy, bilateral salpingectomy, hysterectomy or tubal ligation) at least 3 months prior to first dosing.

### Exclusion Criteria

A subject who met any of the following criteria was excluded from participation in this study:
1. History of clinically significant drug allergy or anaphylaxis, including known hypersensitivity to ibudilast or its formulation components.
2. Positive cotinine test at screening and Day-1.
3. Any clinically significant abnormal finding at physical examination at Screening.
4. Clinically significant abnormal laboratory test results or positive serology test results for HBsAg, HCV antibody, or HIV antigen and antibody at Screening.
5. Clinically significant ECG abnormalities or vital signs abnormalities (systolic blood pressure lower than 90 or over 140 mmHg, diastolic blood pressure lower than 50 or over 90 mmHg, or heart rate less than 50 or over 100 bpm) at Screening.
6. Presence or history of significant gastrointestinal, liver or kidney disease, or surgery that may affect drug bioavailability.
7. Clinical evidence or a history of clinically significant cardiovascular, respiratory, renal, hepatic, gastrointestinal, hematological, neurologic, endocrine, metabolic, psychiatric disease or other chronic diseases as judged by the Investigator.
8. History of alcohol abuse within one year prior to Screening or regular use of alcohol within 6 months prior to Screening that exceeds 10 units for women or 15 units for men of alcohol per week (1 unit=340 mL of beer 5%, 140 mL of wine 12%, or 45 mL of distilled alcohol 40%); and/or history of drug abuse within one year prior to Screening or recreational use of soft drugs (such as marijuana) within 1 month or hard drugs (such as cocaine, phencyclidine [PCP], crack, opioid derivatives including heroin, and amphetamine derivatives) within 3 months prior to Screening.
9. Positive results on urine drug and alcohol screen at Screening visit and Day −1.
10. Platelets<100×10⁹/L at Screening or history of thrombocytopenia
11. Currently pregnant or nursing.
12. Donation of plasma within 7 days prior to first dosing or donation or loss of 500 mL or more of whole blood within 8 weeks prior to first dosing or have poor peripheral venous access.
13. Currently participating, or has participated in a study with an investigational or marketed compound or device within 30 days or 5 half-lives, whichever is longer, prior to first dosing or has participated in a study with a biological product within 90 days or 5 half-lives, whichever is longer, prior to first dosing.
14. Use of medications within the timeframes specified below:
   Depot injection or implant of any drug (with the exception of hormonal contraceptives) for 3 months prior to first dosing and throughout the study;
   Live attenuated vaccines for 1 month prior to first dosing and throughout the study, or any other vaccine, including COVID-19 vaccine, for 14 days prior to first dosing and throughout the study;
   Any drug known to induce or inhibit hepatic drug metabolism (with the exception of hormonal contraceptives), including St. John's wort, for 30 days prior to first dosing and throughout the study;
   Prescription medications for 14 days prior to first dosing and throughout the study;
   Over-the-counter (OTC) medications and natural health products (including herbal remedies, homeopathic and traditional medicines, probiotics, food supplements such as vitamins, minerals, amino acids, essential fatty acids, and protein supplements used in sports) for 7 days prior to first dosing (with the exception of the occasional use of acetaminophen up to 2 g daily) and throughout the study;
   Herbal laxative supplement (e.g., Cassia Senna) or any product that may affect drug absorption and distribution for 14 days prior to first dosing and throughout the study.
15. For Part 2 only: Subjects who cannot complete the high-fat meal within specified time and strict vegetarians/vegans.
16. For Part 3 only: Subjects who cannot eat breakfast in the morning.
17. Any reason which, in the opinion of the Investigator, would prevent the subject from participating in the study.
18. Any subject who has taken ibudilast previously.

### Results

### Part 1 Results Summary

| Prototype | Tₘₐₓ (h) | Cₘₐₓ (ng/mL) | AUC₀₋ₜ (h*ng/mL) | T_{1/2} (h) |
|---|---|---|---|---|
| CAP1 | 4.3 | 38.9 | 1015.3 | 42.4 |
| CAP2 | 3.0 | 68.3 | 1245.6 | 53.4 |
| CAP3 | 3.0 | 67.4 | 1093.4 | 45.9 |

### Part 2 Results Summary

| Prototype | Tₘₐₓ | Cₘₐₓ | AUC₀₋ₜ | T_{1/2} |
|---|---|---|---|---|
| condition | (h) | (ng/mL) | (h*ng/mL) | (h) |
| MRF fasted | 3.6 | 79.8 | 1475.1 | 41.7 |
| MRF fed | 6.5 | 102.2 | 1490.1 | 37.9 |
| IRC fasted | 4.6 | 73.4 | 1461.5 | 45.7 |

Notably, under fed conditions, CAP3 provided a higher Tₘₐₓ compared with IRC under fasted conditions. However, AUC values were similar for the subjects between these two groups.

### Part 3 Results Summary

Day 10 (after 50 mg BID dosing for 10 days)

| Prototype | Tₘₐₓ | Cₘₐₓ | AUC₀₋ₜ | T_{1/2} |
|---|---|---|---|---|
| condition | (h) | (ng/mL) | (h*ng/mL) | (h) |
| CAP3 with BR | 4.4 | 95.5 | 2057.9 | 49.7 |
| CAP3 without BR | 3.0 | 120.7 | 3279.9 | 55.4 |

These results confirm the food effect for CAP3 observed in Part 2.

It was previously reported that human patients administered IRC with an average meal (not high fat) according to the schedule in Table A below provided PK results shown in Table B below. These results suggest that IRC has little food effect.

TABLE A

| Study Day | Dose and Dosing Schedule |
|---|---|
| Days 1-2 | 20 mg BID |
| Days 3-4 | 30 mg BID |
| Days 5-6 | 40 mg BID |
| Days 7-13 | 50 mg BID |
| Day 14 | 50 mg QAM |
| Days 15-16 | No medication administered |

TABLE B

| | Tₘₐₓ (h) | Cₘₐₓ (ng/mL) | AUC₀₋₂₄ (h*ng/mL) | T_{1/2} (h)* |
|---|---|---|---|---|
| IRC (50 mg) | 4.9 | 115 | 1613 | 20.8 |
| With meal | | | | |

*sample was collected up to post-dose 48 hours

The use of the term “or” in the claims is used to mean “and/or” unless explicitly indicated to refer to alternatives only or the alternatives are mutually exclusive, although the disclosure supports a definition that refers to only alternatives and “and/or.”

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A pharmaceutical composition comprising extended-release pellets and optionally delayed release pellets, wherein:
each of the extended-release pellets and each of the delayed release pellets comprises a drug pellet and an outer coating;
the outer coating of the extended-release pellets is an extended-release outer coating;
the outer coating of the delayed release pellets is a delayed release outer coating;
the drug pellet comprises about 15 wt. % to about 25 wt. % ibudilast, about 60 wt. % to about 70 wt. % sugar sphere, about 5 wt. % to about 10 wt. % hydroxypropyl methylcellulose, and about 4 wt. % to about 8 wt. % silicon dioxide;
the extended-release outer coating comprises about 20 wt. % to about 55 wt. % ethyl cellulose, about 20 wt. % to about 55 wt. % hydroxypropyl methylcellulose, about 1 wt. % to about 4 wt. % triethyl citrate, and about 20 wt. % to about 25 wt. % talc, wherein the weight percentages of ethyl cellulose, hydroxypropyl methylcellulose, triethyl citrate, and talc are based on total weight of the extended-release outer coating; and
the delayed release outer coating comprises about 55 wt. % to about 65 wt. % copolymer of methacrylic acid and ethyl acrylate, about 3 wt. % to about 9 wt. % triethyl citrate, and about 25 wt. % to about 35 wt. % talc, wherein the weight percentages of the copolymer of methacrylic acid and ethyl acrylate, triethyl citrate, and talc are based on total weight of the delayed release outer coating.

2. The pharmaceutical composition of claim 1, wherein ethyl cellulose and hydroxypropyl methylcellulose are present in the extended-release outer coating in a ratio of wt. % ethyl cellulose to wt. % hydroxypropyl methylcellulose of 2:1 to 1:2.

3. The pharmaceutical composition of claim 1, wherein ethyl cellulose and triethyl citrate are present in the extended-release outer coating in a ratio of wt. % ethyl cellulose to wt. % triethyl citrate of 100:7.

4. The pharmaceutical composition of claim 1, wherein the drug pellet comprises about 22 wt. % ibudilast, about 65 wt. % sugar sphere, about 7 wt. % hydroxypropyl methylcellulose, and about 6 wt. % silicon dioxide, wherein the weight percentages of ibudilast, sugar sphere, hydroxypropyl methylcellulose, and silicon dioxide are based on total weight of the drug pellet.

5. The pharmaceutical composition of claim 1, wherein:
(i) the drug pellet is present in the extended-release pellet in an amount of about 90 wt. % to about 97 wt. %, and the extended-release outer coating is present in the extended-release pellet in an amount of about 3 wt. % to about 10 wt.;
(ii) the drug pellet is present in the extended-release pellet in an amount of about 90.9 wt. %, and the extended-release outer coating is present in the extended-release pellet in an amount of about 9.1 wt. %;
(iii) the drug pellet is present in the extended-release pellet in an amount of about 93.0 wt. %, and the extended-release outer coating is present in the extended-release pellet in an amount of about 7.0 wt. %;
(iv) the drug pellet is present in the extended-release pellet in an amount of about 94.0 wt. %, and the extended-release outer coating is present in the extended-release pellet in an amount of about 6.0 wt. %;
(v) the drug pellet is present in the extended-release pellet in an amount of about 94.3 wt. %, and the extended-release outer coating is present in the extended-release pellet in an amount of about 5.7 wt. %; or
(vi) the drug pellet is present in the extended-release pellet in an amount of about 94.6 wt. %, and the extended-release outer coating is present in the extended-release pellet in an amount of about 5.4 wt. %.

6.-10. (canceled)

11. The pharmaceutical composition of claim 5, wherein:
(i) the extended-release outer coating comprises about 37.4 wt. % ethyl cellulose, about 37.4 wt. % hydroxypropyl methylcellulose, about 2.6 wt. % triethyl citrate, and about 22.5 wt. % talc;
(ii) the extended-release outer coating comprises about 49.5 wt. % ethyl cellulose, about 24.8 wt. % hydroxypropyl methylcellulose, about 3.5 wt. % triethyl citrate, and about 22.2 wt. % talc; or
(iii) the extended-release outer coating comprises about 25.3 wt. % ethyl cellulose, about 50.3 wt. % hydroxypropyl methylcellulose, about 1.7 wt. % triethyl citrate, and about 22.7 wt. % talc;
wherein the weight percentages of ethyl cellulose, hydroxypropyl methylcellulose, triethyl citrate, and talc are based on total weight of the extended-release outer coating.

12.-13. (canceled)

14. The pharmaceutical composition of claim 1, comprising extended-release pellets and delayed release pellets.

15. The pharmaceutical composition of claim 14, wherein the delayed release outer coating comprises about 62.4 wt. % of the copolymer of methacrylic acid and ethyl acrylate, 6.3 wt. % of triethyl citrate, and 31.3 wt. % talc, wherein the weight percentages of the copolymer of methacrylic acid and ethyl acrylate, triethyl citrate, and talc are based on total weight of the delayed release outer coating.

16. The pharmaceutical composition of claim 14, wherein:
(i) the drug pellet is present in the delayed release pellet in an amount of about 75 wt. % to about 90 wt. %, and the delayed release outer coating is present in the delayed release pellet in an amount of about 10 wt. % to about 25 wt. %;
(ii) the drug pellet is present in the delayed release pellet in an amount of about 80 wt. %, and the delayed release outer coating is present in the delayed release pellet in an amount of about 20 wt. %; or
(iii) the drug pellet is present in the delayed release pellet in an amount of about 87 wt. %, and the delayed release outer coating is present in the delayed release pellet in an amount of about 13 wt. %.

17.-18. (canceled)

19. The pharmaceutical composition of claim 1, wherein the extended-release pellets and delayed release pellets are present in the pharmaceutical composition in a ratio of number of extended-release pellets to number of delayed release pellets of 95:5 to 30:70.

20. The pharmaceutical composition of claim 19, wherein the extended-release pellets and delayed release pellets are present in the pharmaceutical composition in a ratio of number of extended-release pellets to number of delayed release pellets of about 80:20.

21. The pharmaceutical composition of claim 19, wherein the extended-release pellets and delayed release pellets are present in the pharmaceutical composition in a ratio of number of extended-release pellets to number of delayed release pellets of about 50:50.

22. The pharmaceutical composition of claim 1, with a pharmacokinetic profile having a higher Tₘₐₓ value compared to a Tₘₐₓ value of an intermediate release formulation.

23. The pharmaceutical composition of claim 22, wherein the pharmacokinetic profile has similar a AUC value compared to AUC value of the intermediate release formulation.

24. The pharmaceutical composition of claim 1 comprising no delayed release pellets.

25. The pharmaceutical composition of claim 1 in the form of a capsule.

26. The pharmaceutical composition of claim 25, wherein the capsule has about 10 mg to about 200 mg of ibudilast.

27. The pharmaceutical composition of claim 26, wherein the capsule has about 50 mg of ibudilast.

28. The pharmaceutical composition of claim 1, wherein ibudilast is the sole active agent.

29.-57. (canceled)
